(19) United States
(12) Patent Application Publication
(10) Pub. No.: US 2023/0149396 A1
(43) Pub. Date: May 18, 2023

(54) ORAL SOLID PREPARATIONS

(71) Applicant: Takeda Pharmaceutical Company Limited, Osaka-shi, Osaka (JP)

(72) Inventors: Tetsuya SUEHARA, Osaka-shi, Osaka (JP); Miyuki HOHOKABE, Fujisawa-shi, Kanagawa-ken (JP); Kazuki HARADA, Osaka-shi, Osaka (JP)

(73) Assignee: Takeda Pharmaceutical Company Limited, Osaka-shi, Osaka (JP)

(21) Appl. No.: 17/905,616

(22) PCT Filed: Mar. 3, 2021
(86) PCT No.: PCT/IB2021/000115

§ 371 Date: Sep. 2, 2022

(30) Foreign Application Priority Data
Mar. 4, 2020 (JP) 2020-037177

(51) Int. Cl. A61K 31/50; A61K 9/20; A61K 9/28

(52) U.S. Cl. CPC A61K 31/50; A61K 9/2054; A61K 9/2018; A61K 9/2866; A61K 9/2813; A61K 9/2095

(57) ABSTRACT

Disclosed herein are oral solid preparations comprising a D-amino acid oxidase (DA AO) inhibitor, a low-substituted hydroxypropyl cellulose (L-HPC), and an additive, wherein the DAAO inhibitor is a pyridazinone derivative, methods for producing the same, and methods of using the same m the prevention or treatment of diseases preventable or treatable using DAAO inhibitors.

This application claims the benefit of priority to Japanese Patent Application No. 2020-037177, filed Mar. 4, 2020, the contents of which are incorporated by reference herein in their entirety.

The present disclosure relates to oral solid preparations comprising a D-amino acid oxidase (DAAO) inhibitor, a low-substituted hydroxypropyl cellulose (L-HPC), and an additive, wherein the DAAO inhibitor is a pyridazinone derivative, methods for producing the same, and uses of the same.

WO 2013/027000, WO 2015/132608, WO 2013/073577, WO 2014/096757, and WO 2019/076329 disclose DAAO inhibitors which are pyridazinone derivatives. DAAO inhibitors have the effect of reducing the activity of DAAO, which is an enzyme that removes D-serine (D-SER) from the synaptic cleft. DAAO inhibitors may be effective for the prevention and/or treatment of schizophrenia, schizophreniform disorder, schizoaffective disorder, cognitive impairment, pain, ataxia disorder, and the like.

The inventors of the present disclosure have conducted various studies on oral solid preparations comprising DAAO inhibitors that are pyridazinone derivatives. The inventors found that these oral solid preparations are unstable and exhibit a dissolution delay after storage at high humidity. This storage instability reduces the dissolution of the oral solid preparation in the digestive tract, reduces the amount of the active ingredient absorbed in the body, and reduces drug efficacy. Therefore, an object of the present disclosure is to provide more storage stable oral solid preparations of DAAO inhibitors that are pyridazinone derivatives.

As a result of diligent research to solve the above stability problem, the inventors of the present disclosure determined that it is possible to provide an oral solid preparation having excellent storage stability by using low-substituted hydroxypropyl cellulose. These oral solid preparations may be useful for treating diseases treatable or preventable by DAAO inhibitors, such as schizophrenia, ataxic disorder, and the like.

Some embodiments of the present disclosure provide an oral solid preparation comprising a DAAO inhibitor, which is a pyridazinone derivative, as an active ingredient, wherein the oral solid preparation possesses desirable storage stability properties. For example, oral solid preparations of the present disclosure may exhibit excellent active ingredient dissolution properties even after storage at high humidity, for example, at 90% RH, for example, for 2 weeks. Furthermore, oral solid preparations of the present disclosure comprising a high content (for example, 50% by weight or more) of a DAAO inhibitor may exhibit excellent dissolution properties post-storage. The high DAAO inhibitor content may enable miniaturization of the oral solid preparation, potentially improving patient compliance.

Non-limiting examples of DAAO inhibitors that are pyridazinone derivatives used in the present disclosure include the compounds described in WO 2013/027000, WO 2015/132608, WO 2013/073577, WO 2014/096757, WO 2019/076329, and the like.

Disclosed herein is an oral solid preparation comprising a D-amino acid oxidase (DAAO) inhibitor, a low-substituted hydroxypropyl cellulose (L-HPC), and an additive, wherein the DAAO inhibitor is a pyridazinone derivative.

In some embodiments, the oral solid preparation is a tablet. In some embodiments, the oral solid preparation is a sugar-coated tablet. In some embodiments, the oral solid preparation is a film-coated tablet.

In some embodiments, the total weight of the oral solid preparation per preparation unit is in the range of 100 mg to 2000 mg.

In some embodiments, the oral solid preparation comprises 10 mg to 1000 mg of the DAAO inhibitor per preparation unit. In some embodiments, the oral solid preparation comprises 50 mg to 600 mg of the DAAO inhibitor per preparation unit. In some embodiments, the oral solid preparation comprises 10 mg of the DAAO inhibitor per preparation unit. In some embodiments, the oral solid preparation comprises 25 mg of the DAAO inhibitor per preparation unit. In some embodiments, the oral solid preparation comprises 50 mg of the DAAO inhibitor per preparation unit. In some embodiments, the oral solid preparation comprises 100 mg of the DAAO inhibitor per preparation unit. In some embodiments, the oral solid preparation comprises 125 mg of the DAAO inhibitor per preparation unit. In some embodiments, the oral solid preparation comprises 250 mg of the DAAO inhibitor per preparation unit.

In some embodiments, the DAAO inhibitor is chosen from compounds of Formula (I) as disclosed herein and pharmaceutically acceptable salts thereof.

In some embodiments, the DAAO inhibitor is chosen from 4-hydroxy-6-{2-[4-(trifluoromethyl)phenyl]ethyl}pyridazine-3(2H)-one (“Compound (A)”) and pharmaceutically acceptable salts thereof. In some embodiments, the DAAO inhibitor is Compound (A). In some embodiments, Compound (A) is in the form of a solvate. In some embodiments, Compound (A) is in the form of a non-solvate. In some embodiments, Compound (A) is in the form of a crystalline form of Compound (A). In some embodiments, Compound (A) is in the form of an amorphous form of Compound (A).

In some embodiments, the oral solid preparation comprises 10 mg to 1000 mg of at least one compound chosen from Compound (A) and pharmaceutically acceptable salts thereof per preparation unit. In some embodiments, the oral solid preparation comprises 50 mg to 600 mg of at least one compound chosen from Compound (A) and pharmaceutically acceptable salts thereof per preparation unit. In some embodiments, the oral solid preparation comprises 10 mg of at least one compound chosen from Compound (A) and pharmaceutically acceptable salts thereof per preparation unit. In some embodiments, the oral solid preparation comprises 25 mg of at least one compound chosen from Compound (A) and pharmaceutically acceptable salts thereof per preparation unit. In some embodiments, the oral solid preparation comprises 50 mg of at least one compound chosen from Compound (A) and pharmaceutically acceptable salts thereof per preparation unit. In some embodiments, the oral solid preparation comprises 100 mg of at least one compound chosen from Compound (A) and pharmaceutically acceptable salts thereof per preparation unit. In some embodiments, the oral solid preparation comprises 125 mg of at least one compound chosen from Compound (A) and pharmaceutically acceptable salts thereof per preparation unit. In some embodiments, the oral solid preparation comprises 250 mg of at least one compound chosen from Compound (A) and pharmaceutically acceptable salts thereof per preparation unit.

In some embodiments, the DAAO inhibitor is chosen from compounds of Formula (I)-a as disclosed herein and pharmaceutically acceptable salts thereof.

In some embodiments, the solid oral preparation comprises one type of L-HPC. In some embodiments, the solid oral preparation comprises two or more types of L-HPC.

In some embodiments, the L-HPC comprises 5.0% to 16.0% of a hydroxypropoxy group by dry weight.

In some embodiments, the L-HPC is chosen from L-HPC LH-11, L-HPC LH-21, LH-31, L-HPC LH-22, L-HPC LH-32, and combinations of any of the foregoing. In some embodiments, the L-HPC is L-HPC LH-21.

In some embodiments, the content of L-HPC in the oral solid preparation is 1% to 20% by weight.

In some embodiments, the additive is chosen from fillers, binders, disintegrants, lubricants, glidants, colorants, pH adjusters, surfactants, stabilizers, acidulants, sweeteners, flavors, coating agents, coating additives, and combinations of any of the foregoing. In some embodiments, the additive is chosen from fillers, binders, disintegrants, lubricants, and combinations of any of the foregoing.

In some embodiments, the additive comprises a filler. In some embodiments, the filler is D-mannitol. In some embodiments, the filler is microcrystalline cellulose. In some embodiments, the content of the filler is 10%/o to 65% by weight. In some embodiments, the content of the filler is 10% to 85% by weight. In some embodiments, the content of the filler is 25% to 65% by weight. In some embodiments, the content of the filler is 25% to 85% by weight.

In some embodiments, the additive comprises a binder. In some embodiments, the binder is hydroxypropyl cellulose. In some embodiments, the content of the binder is 0.5% to 20% by weight.

In some embodiments, the additive comprises a coating agent. In some embodiments, the coating agent is chosen from water-soluble film coating agents. In some embodiments, the additive further comprises a coating additive. In some embodiments, the coating additive is chosen from light-shielding agents, colorants, plasticizers, organic acids, and combinations of any of the foregoing.

In some embodiments, the oral solid preparation comprises:
30% to 60% by weight of Compound (A);
3% to 15% by weight of a L-HPC;
15% to 65% by weight of a filler;
1% to 10% Y, by weight of a binder; and
0.2% to 3% by weight of a lubricant.

In some embodiments, the oral solid preparation comprises:
3% to 60% by weight of Compound (A);
3% to 15% by weight of a L-HPC;
25% to 85% by weight of a filler;
1% to 10% by weight of a binder; and
0.2% to 3% by weight of a lubricant.

In some embodiments, the filler is mannitol and microcrystalline cellulose.

In some embodiments, the binder is hydroxypropyl cellulose.

In some embodiments, the lubricant is magnesium stearate.

In some embodiments, the oral solid preparation further comprises a film coating.

In some embodiments, the film coating comprises a coating agent and a coating additive. In some embodiments, the film coating comprises a coating agent and a light-shielding agent. In some embodiments, the film coating comprises a coating agent, a light-shielding agent, and a colorant.

In some embodiments, the coating agent is chosen from cellulose-based polymers. In some embodiments, the coating agent is hydroxypropyl cellulose, hydroxypropylmethyl cellulose, and combinations thereof. In some embodiments, the coating agent is hydroxypropyl cellulose and hydroxypropylmethyl cellulose. In some embodiments, the coating agent is hydroxypropylmethyl cellulose.

In some embodiments, the film coating comprises hydroxypropylmethyl cellulose, titanium dioxide, and hydroxypropyl cellulose.

In some embodiments, when performing a dissolution test for an oral solid preparation of the present disclosure using a paddle method (75 rpm, using 900 mL of a 0.05 mol/L phosphate buffer solution (pH 6.8) comprising 0.5% of sodium dodecyl sulfate (SDS)), 70% or more of the DAAO inhibitor dissolves within 30 minutes.

In some embodiments, when performing a dissolution test for an oral solid preparation of the present disclosure using a paddle method (50 rpm, using 900 mL of a 0.05 mol/L phosphate buffer solution (pH 6.8) comprising 0.1% of sodium dodecyl sulfate (SDS)) after storing for two weeks at 40° C./90% relative humidity, 70% or more of the DAAO inhibitor dissolves within 30 minutes. In some embodiments, the oral solid preparation is placed into a glass bottle and stored without a cap for 2 weeks at 40° C./90% relative humidity before performing the dissolution test.

In some embodiments, when performing a dissolution test for an oral solid preparation of the present disclosure using a paddle method (50 rpm, using 900 mL of a 0.05 mol/L phosphate buffer solution (pH 6.8)) after storing for two weeks at 40° C./90% relative humidity, 70% or more of the DAAO inhibitor dissolves within 30 minutes. In some embodiments, the oral solid preparation is placed into a glass bottle and stored without a cap for 2 weeks at 40° C./90% relative humidity before performing the dissolution test.

In some embodiments, when performing a dissolution test for an oral solid preparation of the present disclosure using a paddle method (50 rpm, using 900 mL of a 0.05 mol/L phosphate buffer (pH 6.8) comprising 0.2% of cetyltrimethylammonium bromide (CTAB)) after storing for two weeks at 40° C./90% relative humidity, 70% or more of the DAAO inhibitor dissolves within 30 minutes. In some embodiments, the oral solid preparation is placed into a glass bottle and stored without a cap for 2 weeks at 40° C./90% relative humidity before performing the dissolution test.

In some embodiments, the oral solid preparation is a specific preparation described herein, for example, in the examples of the present disclosure. In some embodiments, the oral solid preparation has the composition described in Table 2. In some embodiments, the oral solid preparation has the composition described in Table 3. In some embodiments, the oral solid preparation has the composition described in Table 4. In some embodiments, the oral solid preparation has the composition described in Table 5. In some embodiments, the oral solid preparation has the composition described in Table 6. In some embodiments, the oral solid preparation has the composition described in Table 7. In some embodiments, the oral solid preparation has the composition described in Table 8. In some embodiments, the oral solid preparation has the composition described in Table 9. In some embodiments, the oral solid preparation has the composition described in Table 10. In some embodiments, the oral solid preparation has the composition described in Table 11. In some embodiments, the oral solid preparation has the composition described in Table 12. In some embodiments, the oral solid preparation has the composition described in Table 13.

In some embodiments, an oral solid preparation of the present disclosure can be used in combination with one or more other types of drugs.

Also disclosed herein is a method of preparing an oral solid preparation described herein.

In some embodiments, the method comprises:
mixing a DAAO inhibitor and an additive to obtain a mixture;
granulating the mixture to obtain at least one granule;
mixing the at least one granule and a L-HPC to obtain at least one mixed granule; and
compressing the at least one mixed granule.

Also disclosed herein is a method of treating preventing or treating a disease preventable or treatable by a D-amino acid oxidase inhibitor in a mammal in need thereof, the method comprising administering an oral solid preparation described herein to the mammal.

In some embodiments, the disease preventable or treatable by a D-amino acid oxidase inhibitor is chosen from schizophrenia and other mental disorders, dementia and other cognitive disorders, anxiety disorder, mood disorders, sleep disorders, disorders generally first diagnosed in early childhood, childhood or adolescence, pain, neurodegenerative disorders, and ataxic disorders.

In some embodiments, the oral solid preparation is administered in combination with another active pharmaceutical ingredient (i.e., a combination drug). In some embodiments, the oral solid preparation and the combination drug are administered at the same time. In some embodiments, the oral solid preparation and the combination drug are administered at different times. In some embodiments, the oral solid preparation and the combination drug are administered in the same preparation. In some embodiments, the oral solid preparation and the combination drug are administered in different preparations.

### EXAMPLE EMBODIMENTS 1

Without limitation, some embodiments of the disclosure include:
1. An oral solid preparation comprising a D-amino acid oxidase inhibitor, a low-substituted hydroxypropyl cellulose (L-HPC), and an additive, wherein the D-amino acid oxidase inhibitor is a pyridazinone derivative.
2. The oral solid preparation according to Embodiment 1, wherein the additive is chosen from a filler, a binder, a disintegrant, and a lubricant.
3. The oral solid preparation according to Embodiment 1 or 2, wherein the oral solid preparation is a tablet.
4. The oral solid preparation according to any one of Embodiments 1 to 3, wherein the pyridazinone derivative is 4-hydroxy-6-{2-[4-(trifluoromethyl)phenyl]ethyl}pyridazine-3(2H)-one.
5. The oral solid preparation according to any one of Embodiments 1 to 4, wherein the oral solid preparation comprises 10 mg to 1000 mg of a D-amino acid oxidase inhibitor per preparation unit.
6. The oral solid preparation according to Embodiment 1, wherein the L-HPC comprises 5.0% to 16.0% of a hydroxypropoxy group by dry weight.
7. The oral solid preparation according to any one of Embodiments 1 to 6, wherein when performing a dissolution test using a paddle method (75 rpm, using 900 mL of a 0.05 mol/L phosphate buffer solution (pH 6.8) comprising 0.05% of cetyltrimethylammonium bromide (CTAB)), 70% or more of the D-amino acid oxidase inhibitor dissolves within 30 minutes.
8. The oral solid preparation according to any one of Embodiments 1 to 6, wherein when performing a dissolution test using a paddle method (50 rpm, using 900 mL of a 0.05 mol/L phosphate buffer solution (pH 6.8) comprising 0.1% of sodium dodecyl sulfate (SDS)) after storing for two weeks at 40° C./90% relative humidity, 70% or more of the D-amino acid oxidase inhibitor dissolves within 30 minutes.
9. A method for producing an oral solid preparation according to Embodiment 1, comprising:
   a) a step for mixing the D-amino acid oxidase inhibitor and the additive;
   b) a step for granulating the mixture obtained in step a);
   c) a step for mixing the granules obtained in step b) and L-HPC; and
   d) a step for compressing the mixed granules obtained in step c).
10. The method according to Embodiment 9, wherein step a) is a step for mixing the D-amino acid oxidase inhibitor, L-HPC, and the additive.
11. The method according to Embodiment 9 or 10, wherein a high-shear granulation method is used in step b).
12. The oral solid preparation according to Embodiment 1, wherein the oral solid preparation is used to prevent or treat a disease preventable or treatable by a D-amino acid oxidase inhibitor.
13. A method for preventing or treating a disease preventable or treatable by a D-amino acid oxidase inhibitor, comprising administering an oral solid preparation according to Embodiment 1 to a mammal.

### EXAMPLE EMBODIMENTS 2

Without limitation, some embodiments of the disclosure include:
1. An oral solid preparation comprising a DAAO inhibitor, a low-substituted hydroxypropyl cellulose (L-HPC), and an additive, wherein the DAAO inhibitor is a pyridazinone derivative.
2. The oral solid preparation according to Embodiment 1, wherein the oral solid preparation is a tablet.
3. The oral solid preparation according to Embodiment 1 or 2, wherein the oral solid preparation is a film-coated tablet.
4. The oral solid preparation according to any one of Embodiments 1 to 3, wherein the additive is chosen from fillers, binders, disintegrants, lubricants, and combinations of any of the foregoing.
5. The oral solid preparation according to Embodiment 4, wherein the filler is chosen from mannitol, microcrystalline cellulose, starches, and combinations of any of the foregoing.
6. The oral solid preparation according to Embodiment 4 or 5, wherein the filler is mannitol and microcrystalline cellulose.
7. The oral solid preparation according to any one of Embodiments 4 to 6, wherein the content of the filler is 10% to 65% by weight.
8. The oral solid preparation according to any one of Embodiments 4 to 6, wherein the content of the filler is 10% to 85% by weight.
9. The oral solid preparation according to any one of Embodiments 4 to 6, wherein the content of the filler is 25% to 85% by weight.
10. The oral solid preparation according to any one of Embodiments 4 to 9, wherein the binder is hydroxypropyl cellulose.
11. The oral solid preparation according to any one of Embodiments 4 to 10, wherein the content of the binder is 0.5% to 20% by weight.
12. The oral solid preparation according to any one of Embodiments 4 to 11, wherein the lubricant is magnesium stearate.
13. The oral solid preparation according to any one of Embodiments 4 to 12, wherein the content of the lubricant is 0.1% to 5% by weight.
14. The oral solid preparation according to any one of Embodiments 1 to 13, wherein the DAAO inhibitor is chosen from compounds of Formula (I):

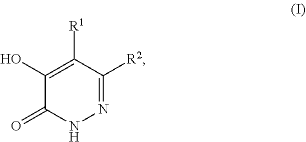

wherein:
R¹ is hydrogen, fluorine, or trifluoromethyl;
R² is chosen from -XYR³ groups;
X and Y are independently chosen from a bond, oxygen, —C(O), —S(O)ₙ groups, —C(O)NR⁴ groups, —S(O)₂NR⁴ groups, —NR⁴ groups,

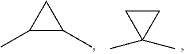

and —CR⁴R⁵— groups, wherein:
   X and Y are not both a bond; and
   when neither X nor Y is a bond, then at least one of X and Y is chosen from —CR⁴R⁵— groups;
n is 0, 1, or 2;
each R⁴ is independently chosen from hydrogen, C₁-C₆alkyl groups, and C₁-C₆ haloalkyl groups;
each R⁵ is independently chosen from hydrogen, C₁-C₆alkyl groups, C₁-C₆haloalkyl groups, and ═CH—;
R³ is chosen from saturated or unsaturated carbocyclic or heterocyclic ring systems of 3 to 10 members, wherein the ring system is optionally substituted by at least one substituent chosen from halogen groups, hydroxyl, cyano, oxo, C₁-C₆alkyl groups, C₂-C₆ alkenyl groups, C₁-C₆haloalkyl groups, C₁-C₆ hydroxyalkyl groups, C₁-C₆alkoxy groups, C₁-C₆ haloalkoxy groups, C₁-C₆alkylthio groups, C₁-C₆alkylsulfinyl groups, C₁-C₆alkylsulfonyl groups, C₁-C₆ alkylcarbonyl groups, C₁-C₆alkylcarbonyloxy groups, C₁-C₆alkoxycarbonyl groups, amino, —CON(R)₂ groups, C₁-C₆ alkyl amino groups, di-(C₁-C₆ alkyl) amino groups, C₃-C₆ cycloalkyl groups, C₃-C₆ cycloalkyloxy groups, C₃-C₆ cycloalkyl methyl groups, —[O]p-(CH₂)q-O—R⁷ groups, and saturated or unsaturated heterocyclic rings of 4 to 6 members optionally substituted by at least one substituent chosen from C₁-C₄ alkyl groups and C₁-C₄ alkoxy groups;
each R⁶ is independently chosen from hydrogen and C₁-C₆ alkyl groups;
p is 0 or 1;
q is 1, 2, 3, or 4; and
R⁷ is chosen from C₁-C₆alkyl groups,
and pharmaceutically acceptable salts thereof.
15. The oral solid preparation according to any one of Embodiments 1 to 14, wherein the DAAO inhibitor is chosen from:
4-hydroxy-6-(2-phenylethyl)pyridazine-3(2H)-one;
6-[2-(4-fluorophenyl)ethyl]-4-hydroxypyridazine-3(2H)-one;
4-hydroxy-6-{2-[5-(trifluoromethyl)pyridin-2-yl]ethyl}pyridazine-3(2H)-one;
6-[(4-chlorobenzyl) sulfanyl]-4-hydroxypyridazine-3(2H)-one;
4-hydroxy-6-{2-[6-(trifluoromethyl)pyridin-3-yl]ethyl}pyridazine-3(2H)-one;
6-[2-(3-fluorophenyl)ethyl]-4-hydroxypyridazine-3(2H)-one;
6-[2-(2-fluorophenyl)ethyl]-4-hydroxypyridazine-3(2H)-one;
6-[2-(3,5-difluorophenyl)ethyl]-4-hydroxypyridazine-3(2H)-one;
6-[2-(3,4-difluorophenyl)ethyl]-4-hydroxypyridazine-3(2H)-one;
4-hydroxy-6-{2-[3-(trifluoromethoxy)phenyl]ethyl}pyridazine-3(2H)-one;
4-hydroxy-6-{2-[3-(trifluoromethyl)phenyl]ethyl}pyridazine-3(2H)-one;
4-hydroxy-6-{2-[5-(trifluoromethyl) pyridin-3-yl]ethyl}pyridazine-3(2H)-one;
6-(2-cyclohexylethyl)-4-hydroxypyridazine-3(2H)-one;
6-(2-cyclopropylethyl)-4-hydroxypyridazine-3(2H)-one;
6-(2-cyclopentylethyl)-4-hydroxypyridazine-3(2H)-one;
4-hydroxy-6-[2-(4-methoxycyclohexyl)ethyl]pyridazine-3(2H)-one;
6-[2-(2,4-difluorophenyl)ethyl]-4-hydroxypyridazine-3(2H)-one;
6-{2-[3-(difluoromethyl)phenyl]ethyl}-4-hydroxypyridazine-3(2H)-one;
6-benzyl-4-hydroxypyridazine-3(2H)-one;
6-[2-(3-chlorophenyl)ethyl]-4-hydroxypyridazine-3(2H)-one;
4-hydroxy-6-(1-phenylcyclopropyl)pyridazine-3(2H)-one; 4-[2-(5-hydroxy-6-oxo-1,6-dihydropyridazine-3-yl)ethyl]benzonitrile;
6-[2-(3-fluoro-4-methylphenyl)ethyl]-4-hydroxypyridazine-3(2H)-one;
6-[2-(4-fluoro-3-methylphenyl)ethyl]-4-hydroxypyridazine-3(2H)-one;
6-[2-(3,4-dimethoxyphenyl)ethyl]-4-hydroxypyridazine-3(2H)-one;
6-[2-(4-chlorophenyl)ethyl]-4-hydroxypyridazine-3(2H)-one;
6-[2-(2-chlorophenyl)ethyl]-4-hydroxypyridazine-3(2H)-one;
4-hydroxy-6-{2-[2-(trifluoromethyl)phenyl]ethyl}pyridazine-3(2H)-one;
6-(4-(difluoromethoxy)phenethyl)-4-hydroxypyridazine-3(2H)-one;
6-(4-(trifluoromethoxy)phenethyl)-4-hydroxypyridazine-3(2H)-one;
6-(3-(difluoromethoxy)phenethyl)-4-hydroxypyridazine-3(2H)-one;
6-[1-(4-fluorophenyl)cyclopropyl]-4-hydroxypyridazine-3(2H)-one;
6-[1-(4-fluorophenyl)ethyl]-4-hydroxypyridazine-3(2H)-one;
4-hydroxy-6-{-[3-(trifluoromethyl)phenyl]ethyl}pyridazine-3(2H)-one;
4-hydroxy-6-{2-[4-(trifluoromethyl)phenyl]ethyl}pyridazine-3(2H)-one;
6-((cyclopropylmethyl)(methyl)amino)-4-hydroxypyridazine-3(2H)-one;
6-((cyclohexylmethyl)(methyl)amino)-4-hydroxypyridazine-3(2H)-one;
6-(3-chlorobenzyl)-4-hydroxypyridazine-3(2H)-one;
6-(4-chlorobenzyl)-4-hydroxypyridazine-3(2H)-one;
6-(cyclohexylmethyl)-4-hydroxypyridazine-3(2H)-one;
6-(4-fluorobenzyl)-4-hydroxypyridazine-3(2H)-one;
6-(2-chloro-6-fluorobenzyl)-4-hydroxypyridazine-3(2H)-one;
6-(2-chlorobenzyl)-4-hydroxypyridazine-3(2H)-one;
6-(3-fluorobenzyl)-4-hydroxypyridazine-3(2H)-one;
6-(2-fluorobenzyl)-4-hydroxypyridazine-3(2H)-one;
6-(4-methylbenzyl)-4-hydroxypyridazine-3(2H)-one;
6-(3-methylbenzyl)-4-hydroxypyridazine-3(2H)-one;
4-hydroxy-6-(3-(trifluoromethyl)benzyl)pyridazine-3(2H)-one;
4-hydroxy-6-[2-(oxane-4-yl)ethyl]pyridazine-3(2H)-one;
6-([(4-fluorophenyl)methyl](methyl)amino)-4-hydroxy-pyridazine-3(2H)-one;
6-[2-(2,6-difluorophenyl)ethyl]-4-hydroxy-pyridazine-3(2H)-one;
6-[2-(2-chloro-6-fluorophenyl)ethyl]-4-hydroxy-pyridazine-3(2H)-one;
6-{[3,5-bis(trifluoromethyl)phenyl]methyl}-4-hydroxypyridazine-3(2H)-one;
6-(1-phenylethyl)-4-hydroxypyridazine-3(2H)-one;
6-(cyclopropylmethyl)-4-hydroxy-2,3-dihydropyridazine-3-one;
4-hydroxy-6-{-[4-(trifluoromethyl)phenyl]cyclopropyl}-2,3-dihydropyridazine-3-one;
6-{2-[2-chloro-4-(trifluoromethyl)phenyl]ethyl}-4-hydroxy-2,3-dihydropyridazine-3-one;
6-{2-[2-fluoro-4-(trifluoromethyl)phenyl]ethyl}-4-hydroxy-2,3-dihydropyridazine-3-one;
6-{2-[3,5-bis(trifluoromethyl)phenyl]ethyl}-4-hydroxy-2,3-dihydropyridazine-3-one;
6-{2-[2,4-bis(trifluoromethyl)phenyl]ethyl}-4-hydroxy-2,3-dihydro-pyridazine-3-one;
6-{2-[3,4-bis(trifluoromethyl)phenyl]ethyl}-4-hydroxy-2,3-dihydropyridazine-3-one;
4-hydroxy-6-(3-methyl-4-(trifluoromethyl)phenethyl)pyridazine-3(2H)-one;
3,4-bis(benzyloxy)-6-((3-chloro-4-(trifluoromethyl)phenyl)ethyl)pyridazine;
4-hydroxy-6-{2-[2-methyl-4-(trifluoromethyl)phenyl]ethyl}-2,3-dihydropyridazine-3-one;
6-{2-[3,5-difluoro-4-(trifluoromethyl)phenyl]ethyl}-4-hydroxy-2,3-dihydropyridazine-3-one; and
6-{2-[3-fluoro-4-(trifluoromethyl)phenyl]ethyl}-4-hydroxy-2,3-dihydropyridazine-3-one, and pharmaceutically acceptable salts of any of the foregoing.
16. The oral solid preparation according to any one of Embodiments 1 to 15, wherein the DAAO inhibitor is chosen from:
6-[2-(4-fluorophenyl)ethyl]-4-hydroxypyridazine-3(2H)-one;
4-hydroxy-6-{2-[4-(trifluoromethyl)phenyl]ethyl}pyridazine-3(2H)-one;
6-(4-chlorobenzyl)-4-hydroxypyridazine-3(2H)-one;
6-(2-fluorobenzyl)-4-hydroxypyridazine-3(2H)-one, and
4-hydroxy-6-{2-[4-(trifluoromethyl)phenyl]ethyl}pyridazine-3(2H)-one, and pharmaceutically acceptable salts of any of the foregoing.
17. The oral solid preparation according to any one of Embodiments 1 to 16, wherein the DAAO inhibitor is chosen from 4-hydroxy-6-{2-[4-(trifluoromethyl)phenyl]ethyl}pyridazine-3(2H)-one and pharmaceutically acceptable salts thereof.
18. The oral solid preparation according to any one of Embodiments 1 to 17, wherein the DAAO inhibitor is 4-hydroxy-6-{2-[4-(trifluoromethyl)phenyl]ethyl}pyridazine-3(2H)-one.
19. The oral solid preparation according to any one of Embodiments 1 to 13, wherein the DAAO inhibitor is chosen from compounds of Formula (I)-a:

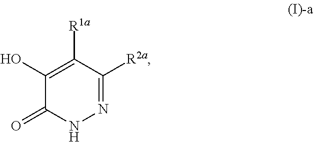

wherein:
R^{1a} is hydrogen, fluorine, or trifluoromethyl;
R^{2a} is chosen from C₂-C₈ alkyl groups, C₃-C₈ cycloalkyl groups, and tetrahydropyranyl, each of which may be optionally substituted with at least one substituent, or R^{2a} is chosen from —NR^{3a}R^{4′} groups;
R^{3a} and R^{4a} are independently chosen from hydrogen and C₁-C₆ alkyl groups, or R^{3a} and R^{4a} form a saturated or unsaturated heterocyclic ring of 4 to 8 members together with a bonded nitrogen atom, and each alkyl group or heterocyclic ring may be optionally substituted by at least one substituent; and
the optional substituents for R^{2a}, R^{3a}, and R^{4a} are independently chosen from halogen groups, hydroxyl, cyano, carboxyl, C₁-C₆alkyl groups, difluoromethyl, trifluoromethyl, C₁-C₆ alkoxy groups, difluoromethoxy, and trifluoromethoxy, provided that the compound is not:
2,3-dihydro-4-hydroxy-6-morpholinopyridazine-3-one, or
6-amino-4-hydroxy-pyridazinone,
and pharmaceutically acceptable salts thereof.
20. The oral solid preparation according to any one of Embodiments 1 to 13 or 19, wherein the DAAO inhibitor is chosen from:
6-ethyl-4-hydroxypyridazine-3(2H)-one;
4-hydroxy-6-(3-methylbutyl)pyridazine-3(2H)-one;
6-cyclopropyl-4-hydroxypyridazine-3(2H)-one;
4-hydroxy-6-(tetrahydro-2H-pyran-4-yl)pyridazine-3(2H)-one;
4-hydroxy-6-(2-methylpropyl)pyridazine-3(2H)-one;
6-cyclopentyl-4-hydroxypyridazine-3(2H)-one;
6-cyclohexyl-4-hydroxypyridazine-3(2H)-one;
4-hydroxy-6-isopropylpyridazine-3(2H)-one;
6-(azetidine-1-yl)-4-hydroxypyridazine-3(2H)-one;
6-(dimethylamino)-4-hydroxypyridazine-3(2H)-one;
4-hydroxy-6-(methyl(propyl)amino)pyridazine-3(2H)-one;
6-(ethyl(methyl)amino)-4-hydroxypyridazine-3(2H)-one;
4-hydroxy-6-(piperidine-1-yl)pyridazine-3(2H)-one;
6-tert-butyl-4-hydroxypyridazine-3(2H)-one; and
4-hydroxy-6-isopropylpyridazine-3(2H)-one, and
pharmaceutically acceptable salts of any of the foregoing.
21. The oral solid preparation according to any one of Embodiments 1 to 20, wherein the oral solid preparation comprises 10 mg to 1000 mg of the DAAO inhibitor per preparation unit.
22. The oral solid preparation according to any one of Embodiments 1 to 21, wherein the oral solid preparation comprises 100 mg to 500 mg of the DAAO inhibitor per preparation unit.
23. The oral solid preparation according to any one of Embodiments 1 to 21, wherein the oral solid preparation comprises 10 mg of the DAAO inhibitor per preparation unit.
24. The oral solid preparation according to any one of Embodiments 1 to 21, wherein the oral solid preparation comprises 25 mg of the DAAO inhibitor per preparation unit.
25. The oral solid preparation according to any one of Embodiments 1 to 21, wherein the oral solid preparation comprises 50 mg of the DAAO inhibitor per preparation unit.
26. The oral solid preparation according to any one of Embodiments 1 to 21, wherein the oral solid preparation comprises 100 mg of the DAAO inhibitor per preparation unit.
27. The oral solid preparation according to any one of Embodiments 1 to 26, wherein the content of the DAAO inhibitor is 1% to 65% by weight.
28. The solid oral preparation according to Embodiment 27, wherein the DAAO inhibitor is chosen from Compound (A) and pharmaceutically acceptable salts thereof.
29. The solid oral preparation according to Embodiment 28, wherein the content of Compound (A) is 3% to 60% by weight.
30. The solid oral preparation according to any one of Embodiments 27 to 29, wherein the oral solid preparation comprises 50 mg of at least one compound chosen from Compound (A) and pharmaceutically acceptable salts thereof.
31. The oral solid preparation according to any one of Embodiments 1 to 26, wherein the content of the DAAO inhibitor is 30% to 65% by weight.
32. The oral solid preparation according to any one of Embodiments 1 to 26, wherein the content of the DAAO inhibitor is 30% to 60% by weight.
33. The oral solid preparation according to any one of Embodiments 1 to 26, wherein the content of the DAAO inhibitor is 20% to 65% by weight.
34. The oral solid preparation according to any one of Embodiments 1 to 26, wherein the content of the DAAO inhibitor is 20% to 60% by weight.
35. The oral solid preparation according to any one of Embodiments 1 to 34, wherein one type of L-HPC is used.
36. The oral solid preparation according to any one of Embodiments 1 to 34, wherein two or more types of L-HPC is used.
37. The oral solid preparation according to any one of Embodiments 1 to 36, wherein the L-HPC comprises 5.0% to 16.0% of a hydroxypropoxy group by dry weight.
38. The oral solid preparation according to any one of Embodiments 1 to 37, wherein the content of L-HPC is 1% to 20% by weight.
39. The oral solid preparation according to any one of Embodiments 1 to 38, wherein the content of L-HPC is 3% to 15% by weight.
40. An oral solid preparation comprising:
   30% to 60% by weight of 4-hydroxy-6-{2-[4-(trifluoromethyl)phenyl]ethyl}pyridazine-3(2H)-one;
   3% to 15% by weight of a L-HPC;
   15% to 65% by weight of a filler;
   1% to 10% by weight of a binder; and
   0.2% to 3% by weight of a lubricant.
41. An oral solid preparation comprising:
   20% to 60% by weight of 4-hydroxy-6-{2-[4-(trifluoromethyl)phenyl]ethyl}pyridazine-3(2H)-one;
   3% to 15% by weight of a L-HPC;
   15% to 85% by weight of a filler;
   1% to 10% by weight of a binder; and
   0.2% to 3% by weight of a lubricant.
42. An oral solid preparation comprising:
   20% to 60% c by weight of 4-hydroxy-6-(2-[4-(trifluoromethyl)phenyl]ethyl)pyridazine-3(2H)-one;
   3% to 15% by weight of a L-HPC;
   25% to 85% by weight of a filler;
   1% to 10% by weight of a binder; and
   0.2% to 3% by weight of a lubricant.
43. The oral solid preparation according to any one of Embodiments 40 to 42, wherein the filler is chosen from mannitol, microcrystalline cellulose, starches, and combinations of any of the foregoing.
44. The oral solid preparation according to any one of Embodiments 40 to 43, wherein the filler is mannitol and microcrystalline cellulose.
45. The oral solid preparation according to any one of Embodiments 40 to 44, wherein the binder is hydroxypropyl cellulose.
46. The oral solid preparation according to any one of Embodiments 40 to 45, wherein the lubricant is magnesium stearate.
47. The oral solid preparation according to any one of Embodiments 40 to 46, wherein the L-HPC is L-HPC LH-21.
48. An oral solid preparation comprising:
   30% to 60% by weight of 4-hydroxy-6-{2-[4-(trifluoromethyl)phenyl]ethyl}pyridazine-3(2H)-one;
   3% to 15% by weight of a L-HPC;
   10% to 50% by weight of mannitol;
   5% to 15% by weight of microcrystalline cellulose;
   1% to 10% by weight of hydroxypropyl cellulose; and
   0.2% to 3% by weight of magnesium stearate.
49. An oral solid preparation comprising.
   20% to 60% by weight of 4-hydroxy-6-(2-[4-(trifluoromethyl)phenyl]ethyl)pyridazine-3(2H)-one;
   3% to 15% by weight of a L-HPC;
   10% to 75% by weight of mannitol;
   5% to 15% by weight of microcrystalline cellulose;
   1% to 10% by weight of hydroxypropyl cellulose; and
   0.2% to 3% by weight of magnesium stearate.
50. An oral solid preparation comprising:
   20% to 60% by weight of 4-hydroxy-6-{2-[4-(trifluoromethyl)phenyl]ethyl}pyridazine-3(2H)-one;
   3% to 15% by weight of a L-HPC;
   15% to 75% by weight of mannitol;
   5% to 15% by weight of microcrystalline cellulose;
   1% to 10% by weight of hydroxypropyl cellulose; and
   0.2% to 3% by weight of magnesium stearate.
51. The oral solid preparation according to any one of Embodiments 1 to 50, further comprising a film coating.
52. The oral solid preparation according to Embodiment 51, wherein the film coating comprises a coating agent and a coating additive.
53. The oral solid preparation according to Embodiment 52, wherein the coating additive is chosen from light-shielding agents, colorants, plasticizers, organic acids, and combinations of any of the foregoing.
54. The oral solid preparation according to Embodiment 51, wherein the film coating comprises a coating agent and a light-shielding agent.
55. The oral solid preparation according to Embodiment 51, wherein the film coating comprises a coating agent, a light-shielding agent, and a colorant.
56. The oral solid preparation according to Embodiment 51, wherein the film coating comprises hydroxypropyl methylcellulose, titanium dioxide, and hydroxypropyl cellulose.
57. The oral solid preparation according to any one of Embodiments 1 to 56, wherein 70% or more of the D-amino acid oxidase inhibitor dissolves within 30 minutes when performing a first dissolution test using a first paddle method.
58. The oral solid preparation according to Embodiment 57, wherein the first paddle method comprises paddling at 75 rpm using 900 mL of a 0.05 mol/L phosphate buffer solution (pH 6.8) comprising 0.05% of cetyltrimethylammonium bromide (CTAB).
59. The oral solid preparation according to any one of Embodiments 1 to 58, wherein 70% or more of the D-amino acid oxidase inhibitor dissolves within 30 minutes when performing a second dissolution test using a second paddle method, wherein the D-amino acid oxidase inhibitor was stored for two weeks at 40° C./90% relative humidity before performing the second dissolution test.
60. The oral solid preparation according to Embodiment 59, wherein the second paddle method comprises paddling at 50 rpm using 900 mL of a 0.05 mol/L phosphate buffer solution (pH 6.8) comprising 0.1% of sodium dodecyl sulfate (SDS).
61. The oral solid preparation according to any one of Embodiments 1 to 60, wherein the oral solid preparation is used to prevent or treat a disease preventable or treatable by a D-amino acid oxidase inhibitor.
62. The oral solid preparation according to Embodiment 61, wherein the disease preventable or treatable by a D-amino acid oxidase inhibitor is chosen from schizophrenia and other mental disorders, dementia and other cognitive disorders, anxiety disorder, mood disorders, sleep disorders, disorders generally first diagnosed in early childhood, childhood or adolescence, pain, neurodegenerative disorders, and ataxic disorders.
63. A method for producing an oral solid preparation according to any one of Embodiments 1 to 62, comprising:
   mixing a D-amino acid oxidase inhibitor and an additive to obtain a mixture;
   granulating the mixture to obtain at least one granule;
   mixing the at least one granule and a L-HPC to obtain at least one mixed granule; and
   compressing the at least one mixed granule.
64. The method according to Embodiment 63, wherein mixing the D-amino acid oxidase inhibitor and the additive further comprises mixing a L-HPC with the D-amino acid oxidase inhibitor and the additive.
65. The method according to Embodiment 63 or 64, wherein granulating the mixture comprises high-shear granulation.
66. A method for preventing or treating a disease preventable or treatable by a D-amino acid oxidase inhibitor, comprising administering an oral solid preparation according to any one of Embodiments 1 to 60 to a mammal in need thereof.
67. The method according to Embodiment 66, wherein the disease preventable or treatable by a D-amino acid oxidase inhibitor is chosen from schizophrenia and other mental disorders, dementia and other cognitive disorders, anxiety disorder, mood disorders, sleep disorders, disorders generally first diagnosed in early childhood, childhood or adolescence, pain, neurodegenerative disorders, and ataxic disorders.

### Definitions

As used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein.

As used herein, the term “active pharmaceutical ingredient” (“API”), “active ingredient,” or “therapeutic agent” refers to a biologically active compound.

As used herein, an “additive” or “preparation additive” refers to a carrier or an excipient that is useful in preparing a pharmaceutical composition. For example, an additive is generally safe and includes carriers and excipients that are generally considered acceptable for mammalian pharmaceutical use. As a non-limiting example, additives may be solid, semi-solid, or liquid materials which in the aggregate can serve as a vehicle or medium for the active ingredient. Some examples of additives are found in Remington's Pharmaceutical Sciences and the Handbook of Pharmaceutical Excipients and include diluents, vehicles, carriers, ointment bases, binders, disintegrates, lubricants, glidants, sweetening agents, flavoring agents, gel bases, sustained release matrices, stabilizing agents, preservatives, solvents, suspending agents, buffers, emulsifiers, dyes, propellants, coating agents, and others.

As used herein, “administration” of an API to a subject (e.g., a mammal) refers to any route (e.g., oral delivery) of introducing or delivering the API to the subject. Administration includes self-administration and the administration by another.

As used herein, a “condition,” “disorder,” or “disease” relates to any unhealthy or abnormal state.

As used herein, a “core tablet” or an “uncoated tablet” refers to a tablet obtained by: adding an additive such as a filler, binder, disintegrant, or lubricant to an API (e.g., a DAAO inhibitor), mixing; and compressing.

As used herein, an “effective amount” or “effective dose” refers to an amount of a molecule that treats, upon single or multiple dose administration, a subject suffering from a condition. An effective amount can be determined by the attending diagnostician through the use of known techniques and by observing results obtained under analogous circumstances. In determining the effective amount, a number of factors are considered by the attending diagnostician, including, but not limited to: the species of the subject; its size, age, and general health; the specific condition, disorder, or disease involved; the degree of or involvement or the severity of the condition, disorder, or disease, the response of the individual subject; the particular compound administered; the mode of administration; the bioavailability characteristics of the preparation administered; the dose regimen selected; the use of concomitant medication; and other relevant circumstances.

As used herein, an amount expressed in terms of “mg of [X],” where [X] is an API, refers to the total amount in milligrams of [X] calculated based on the free base of [X]. When [X] is a pharmaceutically acceptable salt, an equivalent amount of one or more pharmaceutically acceptable salts of Compound (1) based on the weight of free base therein may be present.

As used herein, the term “increase” refers to altering positively by at least 5%, including, but not limited to, altering positively by 5%, altering positively by 10%, altering positively by 25%, altering positively by 30% altering positively by 50%, altering positively by
75%, or altering positively by 100%.

As used herein, a “mammal” refers to domesticated animals (e.g., dogs, cats, and horses) and humans. In some embodiments, the mammal is a human.

As used herein, the term “modulate” refers to altering positively or negatively. Non-limiting example modulations include a 1% change, a 2% change, a 5% change, a 10% change, a 25% change, a 50% change, a 75% change, or a 100% change.

As used herein, the terms “patient” and “subject” are used interchangeably and refer to a mammal, such as, e.g., a human.

As used herein, the term “reduce” refers to altering negatively by at least 5% including, but not limited to, altering negatively by 5%, altering negatively by 10%, altering negatively by 25%, altering negatively by 300%, altering negatively by 50%, altering negatively by 75%, or altering negatively by 100%.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art.

### Oral Solid Preparations

Some embodiments of the present disclosure relate to an oral solid preparation comprising a D-amino acid oxidase inhibitor, a low-substituted hydroxypropyl cellulose (L-HPC), and an additive, wherein the D-amino acid oxidase inhibitor is a pyridazinone derivative.

DAAO inhibitors that are pyridazinone derivatives can be produced using known methods, for example, the methods described in WO 2013/027000, WO 2013/073577, WO 2014/096757, WO 2019/076329, or corresponding methods.

Dosage forms of the oral solid preparation of the present disclosure include, but are not limited to, granules, tablets (for example, core tablets, film-coated tablets), and the like.

In some embodiments, the DAAO inhibitor is chosen from compounds of Formula (I):

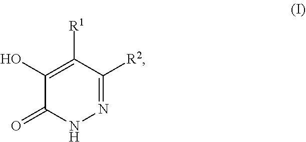

wherein:
R¹ is hydrogen, fluorine, or trifluoromethyl;
R² is chosen from —XYR³ groups;
X and Y are independently chosen from a bond, oxygen, —C(O), —S(O)ₙ groups, —C(O)NR⁴ groups, —S(O)₂NR⁴ groups, —NR⁴ groups,

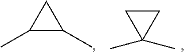

and —CR⁴R⁵-groups, wherein:
   X and Y are not both a bond; and
   when neither X nor Y is a bond, then at least one of X and Y is chosen from —CR⁴R⁵— groups;
n is 0, 1, or 2;
each R⁴ is independently chosen from hydrogen, C₁-C₆alkyl groups, and C₁-C₆ haloalkyl groups;
each R⁵ is independently chosen from hydrogen, C₁-C₆alkyl groups, C₁-C₆haloalkyl groups, and ═CH—;
R³ is chosen from saturated or unsaturated carbocyclic or heterocyclic ring systems of 3 to 10 members, wherein the ring system is optionally substituted by at least one substituent chosen from halogen groups, hydroxyl, cyano, oxo, C₁-C₆alkyl groups, C₂-C₆ alkenyl groups, C₁-C₆haloalkyl groups, C₁-C₆ hydroxyalkyl groups, C₁-C₆ alkoxy groups, C₁-C₆haloalkoxy groups, C₁-C₆alkylthiogroups, C₁-C₆alkylsulfinyl groups, C₁-C₆alkylsulfonyl groups, C₁-C₆ alkylcarbonyl groups, C₁-C₆alkylcarbonyloxy groups, C₁-C₆alkoxycarbonyl groups, amino, —CON(R⁶)₂ groups, C₁-C₆ alkyl amino groups, di-(C₁-C₆ alkyl) amino groups, C₃-C₆ cycloalkyl groups, C₃-C₆ cycloalkyloxy groups, C₃-C₆ cycloalkyl methyl groups, —[O]p-(CH₂)q-O—R⁷ groups, and saturated or unsaturated heterocyclic rings of 4 to 6 members optionally substituted by at least one substituent chosen from C₁-C₄ alkyl groups and C₁-C₄ alkoxy groups;
each R⁶ is independently chosen from hydrogen and C₁-C₆alkyl groups;
p is 0 or 1;
q is 1,2,3, or 4; and
R⁷ is chosen from C₁-C₆alkyl groups,
and pharmaceutically acceptable salts thereof.

In some embodiments, the compound of Formula (I) is chosen from:
4-hydroxy-6-(2-phenylethyl)pyridazine-3(2H)-one;
6-[2-(4-fluorophenyl)ethyl]-4-hydroxypyridazine-3(2H)-one;
4-hydroxy-6-{2-[5-(trifluoromethyl)pyridin-2-yl]ethyl}pyridazine-3(2H)-one;
6-[(4-chlorobenzyl) sulfanyl]-4-hydroxypyridazine-3(2H)-one;
4-hydroxy-6-{2-[6-(trifluoromethyl)pyridin-3-yl]ethyl}pyridazine-3(2H)-one;
6-[2-(3-fluorophenyl)ethyl]-4-hydroxypyridazine-3(2H)-one;
6-[2-(2-fluorophenyl)ethyl]-4-hydroxypyridazine-3(2H)-one;
6-[2-(3,5-difluorophenyl)ethyl]-4-hydroxypyridazine-3(2H)-one;
6-[2-(3,4-difluorophenyl)ethyl]-4-hydroxypyridazine-3(2H)-one;
4-hydroxy-6-{2-[3-(trifluoromethoxy)phenyl]ethyl}pyridazine-3(2H)-one;
4-hydroxy-6-{2-[3-(trifluoromethyl)phenyl]ethyl}pyridazine-3(2H)-one;
4-hydroxy-6-{2-[5-(trifluoromethyl) pyridin-3-yl]ethyl}pyridazine-3(2H)-one;
6-(2-cyclohexylethyl)-4-hydroxypyridazine-3(2H)-one;
6-(2-cyclopropylethyl)-4-hydroxypyridazine-3(2H)-one;
6-(2-cyclopentylethyl)-4-hydroxypyridazine-3(2H)-one;
4-hydroxy-6-[2-(4-methoxycyclohexyl)ethyl]pyridazine-3(2H)-one;
6-[2-(2,4-difluorophenyl)ethyl]-4-hydroxypyridazine-3(2H)-one;
6-{2-[3-(difluoromethyl)phenyl]ethyl}-4-hydroxypyridazine-3(2H)-one;
6-benzyl-4-hydroxypyridazine-3(2H)-one;
6-[2-(3-chlorophenyl)ethyl]-4-hydroxypyridazine-3(2H)-one;
4-hydroxy-6-(1-phenylcyclopropyl)pyridazine-3(2H)-one; 4-[2-(5-hydroxy-6-oxo-1,6-dihydropyridazine-3-yl)ethyl]benzonitrile;
6-[2-(3-fluoro-4-methylphenyl)ethyl]-4-hydroxypyridazine-3(2H)-one;
6-[2-(4-fluoro-3-methylphenyl)ethyl]-4-hydroxypyridazine-3(2H)-one;
6-[2-(3,4-dimethoxyphenyl)ethyl]-4-hydroxypyridazine-3(2H)-one;
6-[2-(4-chlorophenyl)ethyl]-4-hydroxypyridazine-3(2H)-one;
6-[2-(2-chlorophenyl)ethyl]-4-hydroxypyridazine-3(2H)-one;
4-hydroxy-6-{2-[2-(trifluoromethyl)phenyl]ethyl}pyridazine-3(2H)-one;
6-(4-(difluoromethoxy)phenethyl)-4-hydroxypyridazine-3(2H)-one;
6-(4-(trifluoromethoxy)phenethyl)-4-hydroxypyridazine-3(2H)-one;
6-(3-(difluoromethoxy)phenethyl)-4-hydroxypyridazine-3(2H)-one;
6-[1-(4-fluorophenyl)cyclopropyl]-4-hydroxypyridazine-3(2H)-one;
6-[1-(4-fluorophenyl)ethyl]-4-hydroxypyridazine-3(2H)-one;
4-hydroxy-6-{1-[3-(trifluoromethyl)phenyl]ethyl}pyridazine-3(2H)-one;
4-hydroxy-6-{2-[4-(trifluoromethyl)phenyl]ethyl}pyridazine-3(2H)-one;
6-((cyclopropylmethyl)(methyl)amino)-4-hydroxypyridazine-3(2H)-one;
6-((cyclohexylmethyl)(methyl)amino)-4-hydroxypyridazine-3(2H)-one;
6-(3-chlorobenzyl)-4-hydroxypyridazine-3(2H)-one;
6-(4-chlorobenzyl)-4-hydroxypyridazine-3(2H)-one;
6-(cyclohexylmethyl)-4-hydroxypyridazine-3(2H)-one;
6-(4-fluorobenzyl)-4-hydroxypyridazine-3(2H)-one;
6-(2-chloro-6-fluorobenzyl)-4-hydroxypyridazine-3(2H)-one;
6-(2-chlorobenzyl)-4-hydroxypyridazine-3(2H)-one;
6-(3-fluorobenzyl)-4-hydroxypyridazine-3(2H)-one;
6-(2-fluorobenzyl)-4-hydroxypyridazine-3(2H)-one;
6-(4-methylbenzyl)-4-hydroxypyridazine-3(2H)-one;
6-(3-methylbenzyl)-4-hydroxypyridazine-3(2H)-one;
4-hydroxy-6-(3-(trifluoromethyl)benzyl)pyridazine-3(2H)-one;
4-hydroxy-6-[2-(oxane-4-yl)ethyl]pyridazine-3(2H)-one;
6-{[(4-fluorophenyl)methyl](methyl)amino}-4-hydroxy-pyridazine-3(2H)-one;
6-[2-(2,6-difluorophenyl)ethyl]-4-hydroxy-pyridazine-3(2H)-one;
6-[2-(2-chloro-6-fluorophenyl)ethyl]-4-hydroxy-pyridazine-3(2H)-one;
6-([3,5-bis(trifluoromethyl)phenyl]methyl)-4-hydroxypyridazine-3(2H)-one;
6-(1-phenylethyl)-4-hydroxypyridazine-3(2H)-one;
6-(cyclopropylmethyl)-4-hydroxy-2,3-dihydropyridazine-3-one;
4-hydroxy-6-{-[4-(trifluoromethyl)phenyl]cyclopropyl}-2,3-dihydropyridazine-3-one;
6-{2-[2-chloro-4-(trifluoromethyl)phenyl]ethyl}-4-hydroxy-2,3-dihydropyridazine-3-one;
6-{2-[2-fluoro-4-(trifluoromethyl)phenyl]ethyl}-4-hydroxy-2,3-dihydropyridazine-3-one;
6-{2-[3,5-bis(trifluoromethyl)phenyl]ethyl}-4-hydroxy-2,3-dihydropyridazine-3-one;
6-{2-[2,4-bis(trifluoromethyl)phenyl]ethyl}-4-hydroxy-2,3-dihydro-pyridazine-3-one;
6-{2-[3,4-bis(trifluoromethyl)phenyl]ethyl}-4-hydroxy-2,3-dihydropyridazine-3-one;
4-hydroxy-6-(3-methyl-4-(trifluoromethyl)phenethyl)pyridazine-3(2H)-one;
3,4-bis(benzyloxy)-6-((3-chloro-4-(trifluoromethyl)phenyl)ethyl)pyridazine;
4-hydroxy-6-{2-[2-methyl-4-(trifluoromethyl)phenyl]ethyl}-2,3-dihydropyridazine-3-one;
6-{2-[3,5-difluoro-4-(trifluoromethyl)phenyl]ethyl}-4-hydroxy-2,3-dihydropyridazine-3-one; and
6-{2-[3-fluoro-4-(trifluoromethyl)phenyl]ethyl}-4-hydroxy-2,3-dihydropyridazine-3-one.

In some embodiments, the compound of Formula (I) is chosen from:
6-[2-(4-fluorophenyl)ethyl]-4-hydroxypyridazine-3(2H)-one;
4-hydroxy-6-{2-[4-(trifluoromethyl)phenyl]ethyl}pyridazine-3(2H)-one (sometimes abbreviated as “Compound (A)” in the present specification);
6-(4-chlorobenzyl)-4-hydroxypyridazine-3(2H)-one; and
6-(2-fluorobenzyl)-4-hydroxypyridazine-3(2H)-one.

In some embodiments, the DAAO inhibitor is chosen from:
4-hydroxy-6-(2-phenylethyl)pyridazine-3(2H)-one;
6-[2-(4-fluorophenyl)ethyl]-4-hydroxypyridazine-3(2H)-one;
4-hydroxy-6-{2-[5-(trifluoromethyl)pyridin-2-yl]ethyl}pyridazine-3(2H)-one;
6-[(4-chlorobenzyl) sulfanyl]-4-hydroxypyridazine-3(2H)-one;
4-hydroxy-6-{2-[6-(trifluoromethyl)pyridin-3-yl]ethyl}pyridazine-3(2H)-one;
6-[2-(3-fluorophenyl)ethyl]-4-hydroxypyridazine-3(2H)-one;
6-[2-(2-fluorophenyl)ethyl]-4-hydroxypyridazine-3(2H)-one;
6-[2-(3,5-difluorophenyl)ethyl]-4-hydroxypyridazine-3(2H)-one;
6-[2-(3,4-difluorophenyl)ethyl]-4-hydroxypyridazine-3(2H)-one;
4-hydroxy-6-{2-[3-(trifluoromethoxy)phenyl]ethyl}pyridazine-3(2H)-one;
4-hydroxy-6-{2-[3-(trifluoromethyl)phenyl]ethyl}pyridazine-3(2H)-one;
4-hydroxy-6-{2-[5-(trifluoromethyl)pyridin-3-yl]ethyl}pyridazine-3(2H)-one;
6-(2-cyclohexylethyl)-4-hydroxypyridazine-3(2H)-one;
6-(2-cyclopropylethyl)-4-hydroxypyridazine-3(2H)-one;
6-(2-cyclopentylethyl)-4-hydroxypyridazine-3(2H)-one;
4-hydroxy-6-[2-(4-methoxycyclohexyl)ethyl]pyridazine-3(2H)-one;
6-[2-(2,4-difluorophenyl)ethyl]-4-hydroxypyridazine-3(2H)-one;
6-{2-[3-(difluoromethyl)phenyl]ethyl}-4-hydroxypyridazine-3(2H)-one;
6-benzyl-4-hydroxypyridazine-3(2H)-one;
6-[2-(3-chlorophenyl)ethyl]-4-hydroxypyridazine-3(2H)-one;
4-hydroxy-6-(1-phenylcyclopropyl)pyridazine-3(2H)-one; 4-[2-(5-hydroxy-6-oxo-1,6-dihydropyridazine-3-yl)ethyl]benzonitrile;
6-[2-(3-fluoro-4-methylphenyl)ethyl]-4-hydroxypyridazine-3(2H)-one;
6-[2-(4-fluoro-3-methylphenyl)ethyl]-4-hydroxypyridazine-3(2H)-one;
6-[2-(3,4-dimethoxyphenyl)ethyl]-4-hydroxypyridazine-3(2H)-one;
6-[2-(4-chlorophenyl)ethyl]-4-hydroxypyridazine-3(2H)-one;
6-[2-(2-chlorophenyl)ethyl]-4-hydroxypyridazine-3(2H)-one;
4-hydroxy-6-{2-[2-(trifluoromethyl)phenyl]ethyl}pyridazine-3(2H)-one;
6-(4-(difluoromethoxy)phenethyl)-4-hydroxypyridazine-3(2H)-one;
6-(4-(trifluoromethoxy)phenethyl)-4-hydroxypyridazine-3(2H)-one;
6-(3-(difluoromethoxy)phenethyl)-4-hydroxypyridazine-3(2H)-one;
6-[1-(4-fluorophenyl)cyclopropyl]-4-hydroxypyridazine-3(2H)-one;
6-[I-(4-fluorophenyl)ethyl]-4-hydroxypyridazine-3(2H)-one;
4-hydroxy-6-{1-[3-(trifluoromethyl)phenyl]ethyl}pyridazine-3(2H)-one;
4-hydroxy-6-{2-[4-(trifluoromethyl)phenyl]ethyl}pyridazine-3(2H)-one;
6-((cyclopropylmethyl)(methyl)amino)-4-hydroxypyridazine-3(2H)-one;
6-((cyclohexylmethyl)(methyl)amino)-4-hydroxypyridazine-3(2H)-one;
6-(3-chlorobenzyl)-4-hydroxypyridazine-3(2H)-one;
6-(4-chlorobenzyl)-4-hydroxypyridazine-3(2H)-one;
6-(cyclohexylmethyl)-4-hydroxypyridazine-3(2H)-one;
6-(4-fluorobenzyl)-4-hydroxypyridazine-3(2H)-one;
6-(2-chloro-6-fluorobenzyl)-4-hydroxypyridazine-3(2H)-one;
6-(2-chlorobenzyl)-4-hydroxypyridazine-3(2H)-one;
6-(3-fluorobenzyl)-4-hydroxypyridazine-3(2H)-one;
6-(2-fluorobenzyl)-4-hydroxypyridazine-3(2H)-one;
6-(4-methylbenzyl)-4-hydroxypyridazine-3(2H)-one;
6-(3-methylbenzyl)-4-hydroxypyridazine-3(2H)-one;
4-hydroxy-6-(3-(trifluoromethyl)benzyl)pyridazine-3(2H)-one;
4-hydroxy-6-[2-(oxane-4-yl)ethyl]pyridazine-3(2H)-one;
6-{[(4-fluorophenyl)methyl](methyl)amino}-4-hydroxy-pyridazine-3(2H)-one;
6-[2-(2,6-difluorophenyl)ethyl]-4-hydroxy-pyridazine-3(2H)-one;
6-[2-(2-chloro-6-fluorophenyl)ethyl]-4-hydroxy-pyridazine-3(2H)-one;
6-{[3,5-bis(trifluoromethyl)phenyl]methyl}-4-hydroxypyridazine-3(2H)-one;
6-(1-phenylethyl)-4-hydroxypyridazine-3(2H)-one;
6-(cyclopropylmethyl)-4-hydroxy-2,3-dihydropyridazine-3-one;
4-hydroxy-6-{1-[4-(trifluoromethyl)phenyl]cyclopropyl}-2,3-dihydropyridazine-3-one;
6-{2-[2-chloro-4-(trifluoromethyl)phenyl]ethyl}-4-hydroxy-2,3-dihydropyridazine-3-one;
6-{2-[2-fluoro-4-(trifluoromethyl)phenyl]ethyl}-4-hydroxy-2,3-dihydropyridazine-3-one;
6-{2-[3,5-bis(trifluoromethyl)phenyl]ethyl}-4-hydroxy-2,3-dihydropyridazine-3-one;
6-{2-[2,4-bis(trifluoromethyl)phenyl]ethyl}-4-hydroxy-2,3-dihydro-pyridazine-3-one;
6-{2-[3,4-bis(trifluoromethyl)phenyl]ethyl}-4-hydroxy-2,3-dihydropyridazine-3-one;
4-hydroxy-6-(3-methyl-4-(trifluoromethyl)phenethyl)pyridazine-3(2H)-one;
3,4-bis(benzyloxy)-6-((3-chloro-4-(trifluoromethyl)phenyl)ethyl)pyridazine;
4-hydroxy-6-{2-[2-methyl-4-(trifluoromethyl)phenyl]ethyl}-2,3-dihydropyridazine-3-one;
6-{2-[3,5-difluoro-4-(trifluoromethyl)phenyl]ethyl}-4-hydroxy-2,3-dihydropyridazine-3-one; and
6-{2-[3-fluoro-4-(trifluoromethyl)phenyl]ethyl}-4-hydroxy-2,3-dihydropyridazine-3-one, and pharmaceutically acceptable salts of any of the foregoing.

In some embodiments, the DAAO inhibitor is chosen from:
6-[2-(4-fluorophenyl)ethyl]-4-hydroxypyridazine-3(2H)-one;
4-hydroxy-6-{2-[4-(trifluoromethyl)phenyl]ethyl}pyridazine-3(2H)-one;
6-(4-chlorobenzyl)-4-hydroxypyridazine-3(2H)-one; and
6-(2-fluorobenzyl)-4-hydroxypyridazine-3(2H)-one, and
   pharmaceutically acceptable salts of any of the foregoing.

In some embodiments, the DAAO inhibitor is chosen from 4-hydroxy-6-{2-[4-(trifluoromethyl)phenyl]ethyl}pyridazine-3(2H)-one and pharmaceutically acceptable salts thereof. In some embodiments, the DAAO inhibitor is 4-hydroxy-6-{2-[4-(trifluoromethyl)phenyl]ethyl}pyridazine-3(2H)-one.

In some embodiments of the present disclosure, the DAAO inhibitor is chosen from compounds of Formula (I)-a:

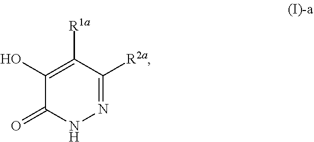

wherein:
R^{1a} is hydrogen, fluorine, or trifluoromethyl;
R^{2a} is chosen from C₂-C₈ alkyl groups, C₃-C₈ cycloalkyl groups, and tetrahydropyranyl, each of which may be optionally substituted with at least one substituent, or R^{2a} is chosen from —NR^{3a}R^{4a} groups;
R^{3a} and R^{4a} are independently chosen from hydrogen and C₁-C₆ alkyl groups, or R^{3a} and R^{4a} form a saturated or unsaturated heterocyclic ring of 4 to 8 members together with a bonded nitrogen atom, and each alkyl group or heterocyclic ring may be optionally substituted by at least one substituent; and
the optional substituents for R^{2a}, R^{3a}, and R^{4a} are independently chosen from halogen groups, hydroxyl, cyano, carboxyl, C₁-C₆alkyl groups, difluoromethyl, trifluoromethyl, C₁-C₆ alkoxy groups, difluoromethoxy, and trifluoromethoxy, provided that the compound is not:
2,3-dihydro-4-hydroxy-6-morpholinopyridazine-3-one, or
6-amino-4-hydroxy-pyridazinone,
and pharmaceutically acceptable salts thereof.

In some embodiments, the compound of Formula (I)-a is chosen from:
6-ethyl-4-hydroxypyridazine-3(2H)-one;
4-hydroxy-6-(3-methylbutyl)pyridazine-3(2H)-one;
6-cyclopropyl-4-hydroxypyridazine-3(2H)-one;
4-hydroxy-6-(tetrahydro-2H-pyran-4-yl)pyridazine-3(2H)-one;
4-hydroxy-6-(2-methylpropyl)pyridazine-3(2H)-one;
6-cyclopentyl-4-hydroxypyridazine-3(2H)-one;
6-cyclohexyl-4-hydroxypyridazine-3(2H)-one;
4-hydroxy-6-isopropylpyridazine-3(2H)-one;
6-(azetidine-1-yl)-4-hydroxypyridazine-3(2H)-one;
6-(dimethylamino)-4-hydroxypyridazine-3(2H)-one;
4-hydroxy-6-(methyl(propyl)amino)pyridazine-3(2H)-one;
6-(ethyl(methyl)amino)-4-hydroxypyridazine-3(2H)-one;
4-hydroxy-6-(piperidine-1-yl)pyridazine-3(2H)-one;
6-tert-butyl-4-hydroxypyridazine-3(2H)-one; and
4-hydroxy-6-isopropylpyridazine-3(2H)-one.

In some embodiments, the DAAO inhibitor is chosen from:
6-ethyl-4-hydroxypyridazine-3(2H)-one;
4-hydroxy-6-(3-methylbutyl)pyridazine-3(2H)-one;
6-cyclopropyl-4-hydroxypyridazine-3(2H)-one;
4-hydroxy-6-(tetrahydro-2H-pyran-4-yl)pyridazine-3(2H)-one;
4-hydroxy-6-(2-methylpropyl)pyridazine-3(2H)-one;
6-cyclopentyl-4-hydroxypyridazine-3(2H)-one;
6-cyclohexyl-4-hydroxypyridazine-3(2H)-one;
4-hydroxy-6-isopropylpyridazine-3(2H)-one;
6-(azetidine-1-yl)-4-hydroxypyridazine-3(2H)-one;
6-(dimethylamino)-4-hydroxypyridazine-3(2H)-one;
4-hydroxy-6-(methyl(propyl)amino)pyridazine-3(2H)-one;
6-(ethyl(methyl)amino)-4-hydroxypyridazine-3(2H)-one;
4-hydroxy-6-(piperidine-1-yl)pyridazine-3(2H)-one;
6-tert-butyl-4-hydroxypyridazine-3(2H)-one; and
4-hydroxy-6-isopropylpyridazine-3(2H)-one, and
   pharmaceutically acceptable salts of any of the foregoing.

Salts of DAAO inhibitors (e.g., Compound (A)) include, but are not limited to, pharmaceutically acceptable salts, such as, for example, salts with inorganic acids, salts with organic acids, and salts with basic or acidic amino acids.

Pharmaceutically acceptable salts of DAAO inhibitors (e.g., compounds of Formula (I) or Formula (I)-a) include, but are not limited to, acid addition salts that can be formed using an inorganic acid or organic acid, such as, for example, hydrochlorides, hydrobromides, benzenesulfonate (besylate), saccharin (for example, monosaccharin), trifluoroacetate, sulfate, nitrate, phosphate, acetate, fumarate, maleate, tartrate, lactate, citrate, pyruvate, succinate, valerate, propanoate, butaneate, malonate, oxalate, 1-hydroxy-2-naphthate (xinafoate), methanesulfonate, or p-toluene sulfonate.

Non-limiting examples of salts with inorganic acids include salts with hydrochloric acid, hydrobromic acid, nitric acid, sulfuric acid, phosphoric acid, and the like.

Non-limiting examples of salts with organic acids include salts with benzoic acid, formic acid, acetic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid, and the like.

Non-limiting examples of salts with basic amino acids include salts with arginine, lysine, ornithine, and the like, and non-limiting examples of salts with acidic amino acids include salts with aspartic acid, glutamic acid, and the like.

In some embodiments, Compound (A) may be in the form of a solvate (for example, a hydrate) or a non-solvate (for example, a non-hydrate).

In some embodiments, Compound (A) may be either an amorphous form or in the form of a crystalline form, for example, in the form of a crystalline form described in WO
2013/027000.

In some embodiments, Compound (A) may be labeled with an isotope (for example, ³H, ¹⁴C, ³⁵S, ¹²⁵I). For example, in some embodiments, deuterium-containing analogs of Compound (A) obtained by converting ¹H to ²H (D) may be used wherever Compound (A) is recited.

Non-limiting examples of the oral solid preparation of the present disclosure include tablets (including sugar-coated tablets and film-coated tablets), pills, granules, capsules, and the like. In some embodiments, the oral solid preparation is a tablet.

In some embodiments, the oral solid preparation comprises 10 mg to 1000 mg of a DAAO inhibitor (for example, Compound (A)) per preparation unit (dosage unit) (for example, per tablet), for example, 50 mg to 600 mg of the DAAO inhibitor per preparation unit, or 100 mg to 500 mg of the DAAO inhibitor per preparation unit.

In some embodiments, the oral solid preparation comprises 50, 100, 125, 200, 250, 300, 400, 500, or 600 mg of a DAAO inhibitor (for example, Compound (A)) per preparation unit (dosage unit) (for example, per tablet). In some embodiments, the oral solid preparation comprises 125 mg of a DAAO inhibitor (for example, Compound (A)) per preparation unit. In some embodiments, the oral solid preparation comprises 250 mg of a DAAO inhibitor (for example, Compound (A)) per preparation unit.

In some embodiments, the content of the DAAO inhibitor (for example, Compound (A)) in the oral solid preparation is 1% to 65% by weight, for example, 20% to 65% by weight, 30% to 65% by weight, 20% to 60% by weight, or 30% to 60% by weight.

The low-substituted hydroxypropyl cellulose (L-HPC) used in the oral solid preparations of the present disclosure is not particularly limited as long as it is used as a pharmaceutical additive. One type of L-HPC may be used alone, or two or more types of L-HPC may be used in combination.

As used herein, a low degree of substitution means that, after drying, the L-HPC comprises 5.0% to 16.0% of a hydroxypropoxy group by dry weight. In some embodiments, the L-HPC comprises 8% to 14%, for example, 11%, of a hydroxypropoxy group by dry weight.

Non-limiting examples of L-HPC include L-HPC LH-11, LH-21, LH-31, LH-22, LH-32 (Shin-Etsu Chemical (Co., Ltd.)), and the like. In some embodiments, the L-HPC is L-HPC LH-21 (Shin-Etsu Chemical (Co., Ltd.)).

In some embodiments, the content of L-HPC in the oral solid preparation is 1% to 20% by weight, for example 3% to 15% by weight.

In some embodiments, the content of L-HPC in the oral solid preparation is 3%, 4%, 5%, 6%, 7%, 8%, 9%, 10%, 11%, 12%, 13%, 14%, or 15% by weight.

In some embodiments, the additive is chosen from pharmaceutically acceptable carriers. Various organic or inorganic carrier substances commonly used as preparation materials may be used as pharmaceutically acceptable carriers, and these substances may be combined in appropriate amounts as, for example, fillers, binders, disintegrants, lubricants, glidants, colorants, pH adjusters, surfactants, stabilizers, acidulants, sweeteners, flavors, coating agents, and coating additives.

In some embodiments, the additive is chosen from fillers, binders, disintegrants, lubricants, and combinations of any of the foregoing.

Non-limiting examples of fillers include: mannitol (for example, D-mannitol {for example, PEARLITOL 50C (product name); Roquette Co.}). microcrystalline cellulose (for example, CEOLUS PH-101 (product name); Asahi Kasei (Co., Ltd.)); starches such as corn starch, potato starch, wheat starch, rice starch, partially pregelatinized starch, pregelatinized starch, and porous starch; anhydrous calcium phosphate; precipitated calcium carbonate; calcium silicate; anhydrous lactose; and lactose hydrate. In some embodiments, the filler is D-mannitol. In some embodiments, the filler is microcrystalline cellulose.

In some embodiments, the content of the filler is 10% to 85% by weight, for example, 10% to 70% by weight, 10% to 66% by weight, 10% to 65% by weight, 25% to 85% by weight, 25% to 70% by weight, 25% to 66% by weight, 25% to 65% by weight, 20% to 85% by weight, 20% to 70% by weight, 20% to 66% by weight, or 20% to 65% by weight.

A binder may be an additive that imparts binding properties between the particles during dry or wet granulation or direct compression, such as, for example, microcrystalline cellulose [for example, microcrystalline cellulose {for example, CEOLUS KG-802 (grade: KG-802) (product name); CEOLUS PH-302 (grade: PH-302) (product name); Asahi Kasei (Co., Ltd.)}, microcrystalline cellulose (granules), microcrystalline cellulose (fine particles)], hydroxypropyl cellulose [for example, grade: L, SL, SSL (product name); NIPPON SODA (CO., LTD.)], hydroxypropylmethyl cellulose [for example, Hypromellose 2910, TC-5 (grade: E, M, R) (product name); Shin-Etsu Chemical (Co., Ltd.)], povidone (polyvinylpyrrolidone), and copovidone. In some embodiments, the binder is hydroxypropyl cellulose.

In some embodiments, the content of the binder is 0.5% to 20% by weight, for example, 1% to 10% by weight.

Non-limiting examples of disintegrants include corn starch, carboxymethyl cellulose, carboxymethyl cellulose calcium, carboxymethyl starch sodium, croscarmellose sodium (for example, Ac-Di-Sol (product name)), crospovidone, low-substituted hydroxypropyl cellulose (L-HPC), hydroxypropyl starch, and partially pregelatinized starch.

In some embodiments, the content of the disintegrant is 1% to 20% by weight, for example, 2% to 15% by weight.

Non-limiting examples of lubricants include magnesium stearate, calcium stearate, talc, sucrose fatty acid ester, and sodium stearyl fumarate. In some embodiments, the lubricant is magnesium stearate.

In some embodiments, the content of the lubricant is 0.1% to 5% by weight, for example, 0.2% to 3% by weight.

Non-limiting examples of glidants include talc, light anhydrous silicic acid, hydrous silicon dioxide, and magnesium aluminometasilicate.

Non-limiting examples of colorants include food pigments such as food yellow 5, food red 2, and food blue 2, edible lake pigments, red ferric oxide, and yellow ferric oxide.

Non-limiting examples of coating agents include a sugar-coating agent, a water-soluble film coating agent, an enteric film coating agent, and a sustained-release film coating agent.

Non-limiting examples of water-soluble film coating agents include cellulose-based polymers such as hydroxypropyl cellulose [for example, grade: L, SL, SSL (product name); NIPPON SODA (CO., LTD.)], hydroxypropylmethyl cellulose [for example, Hypromellose 2910, TC-5 (grade: E, M, R) (product name); Shin-Etsu Chemical (Co., Ltd.)], hydroxyethyl cellulose, and methyl hydroxyethyl cellulose; synthetic polymers such as polyvinyl acetal diethylaminoacetate, aminoalkyl methacrylate copolymer E [EUDRAGIT E (product name)], and polyvinylpyrrolidone; and polysaccharides such as pullulan. The water-soluble film coating agent enables the formation of a water-based film coating that does not affect the dissolution profile of the oral solid preparation.

Coating additives include, but are not limited to, light-shielding agents such as titanium oxide; glidants such as talc; colorants such as red ferric oxide and yellow ferric oxide; plasticizers such as polyethylene glycol (for example, Macrogol 6000), triethyl citrate, castor oil, and polysorbates; and organic acids such as citric acid, tartaric acid, malic acid, and ascorbic acid.

Non-limiting examples of coating agents include OPADRY YELLOW (Colorcon Japan), OPADRY RED (Colorcon Japan), Hypromellose TC-5R (Shin-Etsu Chemical (Co., Ltd.)), titanium dioxide, red ferric oxide, and yellow ferric oxide.

One or more of the above additives may be used in an oral solid preparation of this disclosure at various ratios.

In some embodiments, the oral solid preparation comprises.
30% to 60% by weight of Compound (A);
3% to 15% by weight of a L-HPC;
15% to 65% by weight of a filler;
1% to 10% by weight of a binder; and
0.2% to 3% by weight of a lubricant.

In some embodiments, the oral solid preparation comprises:
3% to 60% by weight of Compound (A);
3% to 15% by weight of a L-HPC;
15% to 85% by weight of a filler;
1% to 10% by weight of a binder; and
0.2% to 3% by weight of a lubricant.

In some embodiments, the oral solid preparation comprises:
20% to 60% by weight of Compound (A);
3% to 15% by weight of a L-HPC;
25% to 85% by weight of a filler;
1% to 10% by weight of a binder; and
0.2% to 3% by weight of a lubricant.

In some embodiments, the oral solid preparation comprises:
20% to 60% by weight of Compound (A);
3% to 15% by weight of a L-HPC;
25% to 66% by weight of a filler;
1% to 10% by weight of a binder; and
0.2% to 3% by weight of a lubricant.

In some embodiments, the filler is mannitol and microcrystalline cellulose.

In some embodiments, the binder is hydroxypropyl cellulose.

In some embodiments, the lubricant is magnesium stearate.

In some embodiments, the oral solid preparation further comprises a film coating. In some embodiments, the film coating comprises a coating agent and a coating additive. In some embodiments, the film coating comprises a coating agent and a light-shielding agent. In some embodiments, the film coating comprises a coating agent, a light-shielding agent, and a colorant. In some embodiments, the coating agent is hydroxypropylmethyl cellulose.

In some embodiments, the film coating comprises hydroxypropylmethyl cellulose, titanium dioxide, and hydroxypropyl cellulose.

In some embodiments, the oral solid preparation comprises:
30% to 60% by weight of Compound (A);
3% to 15% by weight of a L-HPC;
10% to 50% by weight of mannitol;
5% to 15% by weight of microcrystalline cellulose;
1% to 10% by weight of hydroxypropyl cellulose; and
0.2% to 3% by weight of magnesium stearate.

In some embodiments, the oral solid preparation comprises:
3% to 60% by weight of Compound (A);
3% to 15% by weight of a L-HPC;
10% to 75% by weight of mannitol;
5% to 15% by weight of microcrystalline cellulose;
1% to 10% by weight of hydroxypropyl cellulose; and
0.2% to 3% by weight of magnesium stearate.

In some embodiments, the oral solid preparation comprises.
20% to 60% by weight of Compound (A);
3% to 15% by weight of a L-HPC;
15% to 75% by weight of mannitol;
5% to 15% by weight of microcrystalline cellulose;
1% to 10% by weight of hydroxypropyl cellulose; and
0.2% to 3% by weight of magnesium stearate.

In some embodiments, the oral solid preparation comprises:
20%/6 to 60% by weight of Compound (A);
3% to 15% by weight of a L-HPC;
15% to 60% by weight of mannitol;
5% to 15% by weight of microcrystalline cellulose;
1% to 10% by weight of hydroxypropyl cellulose; and
0.2% to 3% by weight of magnesium stearate.

In some embodiments, the oral solid preparation further comprises a film coating. In some embodiments, the film coating comprises a coating agent and a coating additive. In some embodiments, the film coating comprises a coating agent and a light-shielding agent. In some embodiments, the film coating comprises a coating agent, a light-shielding agent, and a colorant. In some embodiments, the coating agent is hydroxypropylmethyl cellulose.

### Dissolution of Oral Solid Preparations

In some embodiments, when performing a dissolution test for an oral solid preparation of the present disclosure using a paddle method (75 rpm, using 900 mL of a 0.05 mol/L phosphate buffer solution (pH 6.8) comprising 0.05% of cetyltrimethylammonium bromide (CTAB)), 70% or more, for example, 75% or more, of the D-amino acid oxidase inhibitor dissolves within 30 minutes.

In some embodiments, when performing a dissolution test for an oral solid preparation of the present disclosure using a paddle method (75 rpm, using 900 mL of a 0.05 mol/L phosphate buffer solution (pH 6.8) comprising 0.5% of sodium dodecyl sulfate (SDS)), 70% or more, for example, 75% or more, of the D-amino acid oxidase inhibitor dissolves within 30 minutes.

In some embodiments, when performing a dissolution test for an oral solid preparation of the present disclosure using a paddle method (50 rpm, using 900 mL of a 0.05 mol/L phosphate buffer solution (pH 6.8) comprising 0.1% of sodium dodecyl sulfate (SDS)) after storing for two weeks at 40° C./90% relative humidity, 70% or more, for example, 75% or more, of the D-amino acid oxidase inhibitor dissolves within 30 minutes. In some embodiments, the oral solid preparation is placed into a glass bottle and stored without a cap for 2 weeks at 40° C./90% relative humidity before performing the dissolution test.

In some embodiments, when performing a dissolution test for an oral solid preparation of the present disclosure using a paddle method (50 rpm, using 900 mL of a 0.05 mol/L phosphate buffer solution (pH 6.8)) after storing for two weeks at 40° C./90% relative humidity, 70% or more, for example, 75% or more, of the D-amino acid oxidase inhibitor dissolves within 30 minutes. In some embodiments, the oral solid preparation is placed into a glass bottle and stored without a cap for 2 weeks at 40° C./90% relative humidity before performing the dissolution test.

In the present specification, the paddle method measures according to the dissolution test method (paddle method, apparatus 2) of the 17th Edition Japanese Pharmacopoeia general test method, with the exception of the conditions specifically mentioned herein.

### Methods of Making Oral Solid Preparations

The oral solid preparation of the present disclosure can be produced using a common method in the field of pharmaceutical preparation.

For example, an oral solid preparation of the present disclosure may be produced by combining operations such as granulation, mixing, compressing (e.g., compression molding), and coating.

In some embodiments, granulation is performed using, for example, a granulator such as a high-shear granulator, a fluid-bed granulator, or a dry granulator.

In some embodiments, mixing is performed using, for example, a mixer such as a V-type mixer or a tumbler mixer.

In some embodiments, compression (compression molding) is performed by, for example, pressing tablets generally at a compression force of 0.3 to 35 kN using a single tablet press, a rotary tablet press, or the like.

In some embodiments, coating is performed, for example, using a film coater along with a coating agent and a coating additive described above.

In some embodiments, when the oral solid preparation of the present disclosure is a tablet, the tablet is film-coated to improve ingestibility, preparation hardness, and the like.

Non-limiting examples of coating agents and coating additives used for film coatings include those similar to the materials described above.

In some embodiments, when the tablet of the present disclosure is film-coated, the film coating layer is formed at a ratio of 1 to 10 parts by weight, for example, 2 to 6 parts by weight, with respect to 100 parts by weight of the tablet.

In some embodiments, when an oral solid preparation of the present disclosure is a film-coated tablet, the content of the DAAO inhibitor (for example, Compound (A)), the L-HPC, and the additive in the uncoated tablet before film coating is within a range described above.

For example, an oral solid preparation of the present disclosure can be produced according to the following production steps. In some embodiments, each raw material in the production steps below is used so that the content in the finally obtained oral solid preparation is present in an amount described above.

In some embodiments, a method for producing an oral solid preparation of the present disclosure comprises:
mixing a DAAO inhibitor and an additive to obtain a mixture;
granulating the mixture to obtain at least one granule;
mixing the at least one granule and a L-HPC to obtain at least one mixed granule; and
compressing the at least one mixed granule.

In some embodiments, the DAAO inhibitor (for example, Compound (A)) and the additive are mixed. In some embodiments, the additive is chosen from fillers, binders, disintegrants, lubricants, and combinations of any of the foregoing. In some embodiments, mixing is performed using, for example, a mixer such as a V-type mixer or a tumbler mixer. In some embodiments, mixing is performed using a granulator such as a high-shear granulator, a fluid-bed granulator, or a dry granulator.

In some embodiments, the oral solid preparation comprises the L-HPC in both a granulated portion and a non-granulated portion to improve a dissolution property. That is, in some embodiments, the method comprises mixing the DAAO inhibitor, the L-HPC, and the additive. In some embodiments, when the L-HPC is contained in both a granulated portion and a non-granulated portion of the oral solid preparation, 10% to 75% by weight, for example, 40% to 60% by weight, of the total amount of the L-HPC is contained in the granulated portion.

In some embodiments, the mixture is granulated using a binder (for example, hydroxypropyl cellulose) and milled as desired. In some embodiments, granulation can be performed using, for example, a high-shear granulation method (high-shear granulation method), a fluidized bed granulation method (fluidized bed granulation method), a dry granulation method (dry granulation method), or the like. In some embodiments, when using the fluidized bed granulation method, the mixture is granulated while spraying a liquid in which a binder is dissolved or dispersed in a solvent or a dispersion medium (for example, water), dried, and milled as necessary to obtain granules. In some embodiments, when using the high-shear granulation method, a liquid such as water is added while stirring the mixture comprising a binder, and granulation, drying, and as necessary, milling is performed to obtain granules. In some embodiments, the high-shear granulation method is employed to increase the content of the DAAO inhibitor.

In some embodiments, the at least one granule is mixed with a L-HPC and, optionally, an additive (for example, a lubricant (for example, magnesium stearate)) to obtain at least one mixed granule. As used herein, a “mixed granule” refers to a mixture containing a granule and a L-HPC. In some embodiments, mixing is performed using, for example, a mixer such as a V-type mixer or a tumbler mixer.

In some embodiments, compression (compression molding) is performed by, for example, compressing the at least one mixed granule at a compression force of 3 to 35 kN using a single tableting press, a rotary tablet press, or the like. Additionally, the oral solid preparation of the present disclosure can be produced by drying, if desired.

In some embodiments, a film-coated tablet is obtained by spraying a film-coating solution or suspension onto a core tablet (i.e., an uncoated tablet) obtained by a method described above.

In some embodiments, a film-coated tablet can be produced by, for example, using a film coater or the like to spray an aqueous solution or suspension of a film coating agent (for example, a film coating agent such as Hypromellose 2910, a plasticizer such as Macrogol 6000, and a mixture of pigments such as titanium oxide, red ferric oxide, and yellow ferric oxide) on a core tablet obtained using a method describe above and covering the core tablet.

In some embodiments, the oral solid preparation of the present disclosure is a tablet comprising 75% to 100% by weight, for example, 80% to 98% by weight, for example, 85% to 95% by weight of the at least one granule.

As used herein, granules refer to particles having substantially the same shape and size that are obtained by granulating a raw material such as powder, clumps, a solution or melted liquid using a wet granulation method, a dry granulation method, a heat granulation method, or the like.

In some embodiments, the weight of the oral solid preparation of the present disclosure per preparation unit (dosage unit) (for example, per tablet) is 50 to 2000 mg, for example, 100 to 1000 mg.

### Therapeutic Methods

Oral solid preparations of the present disclosure may be beneficial as medicines, particularly medicines for inhibiting the enzyme D-amino acid oxidase (DAAO). Because oral solid preparations of the present disclosure may exhibit low toxicity and few side effects, they can be used as medicines to prevent or treat diseases that are preventable or treatable by DAAO inhibitors in mammals (for example, humans, cows, horses, pigs, dogs, cats, monkeys, mice, and rats, and for example, humans).

Diseases preventable or treatable by DAAO inhibitors include, but are not limited to, schizophrenia and other mental disorders (for example, mental disorders and mental illness), dementia and other cognitive disorders, anxiety disorder (for example, generalized anxiety disorder), mood disorders (for example, depressive disorder, major depressive disorder, bipolar disorders including bipolar disorders I and II, bipolar mania, and bipolar depression), sleep disorders, disorders generally first diagnosed in early childhood, childhood or adolescence (for example, attention deficit disorder, autism spectrum disorder, and destructive behavioral disorder), pain (for example, neuropathic pain), neurodegenerative disorders (for example, Parkinson's disease or Alzheimer's disease), ataxic disorders (particularly Friedreich's ataxia or spinocerebellar ataxia), and the like.

Non-limiting examples of diseases preventable or treatable by DAAO inhibitors include positive symptoms of schizophrenia, schizophreniform disorder, or schizoaffective disorder (for example, voices or hallucinations), cognitive disorders (for example, dementia and learning disability) and pain (for example, neuropathic pain).

The present disclosure also provides a method for treating at least one symptom or condition associated with schizophrenia, schizophreniform disorder, schizoaffective disorder and other mental disorders (for example, mental disorders and mental illness), dementia and cognitive disorders, anxiety disorder (for example, generalized anxiety disorder), mood disorders (for example, depressive disorder, major depressive disorder, bipolar disorders including bipolar disorders I and II, bipolar mania, and bipolar depression), sleep disorders, disorders generally first diagnosed in early childhood, childhood or adolescence (for example, attention deficit disorder, autism spectrum disorder, and destructive behavioral disorder), pain (for example, neuropathic pain), neurodegenerative disorders (for example, Parkinson's disease or Alzheimer's disease), ataxic disorder (particularly Friedreich's ataxia or spinocerebellar ataxia), comprising administering an oral solid preparation of the present disclosure comprising a therapeutically effective amount of a DAAO inhibitor to a mammal in need thereof.

In some embodiments, the at least one symptom or condition is chosen from positive and negative psychological symptoms commonly associated with anxiety, agitation, hostility, panic, eating disorders, emotional symptoms, mood symptoms, mental disorders, and neurodegenerative disorders.

In some embodiments, an oral solid preparation of the present disclosure is orally administered to a mammal. In some embodiments, an oral solid preparation of the present disclosure can be safely administered orally to a mammal.

In some embodiments, the oral solid preparation comprises an effective amount of the DAAO inhibitor (for example, Compound (A)) as an active pharmaceutical ingredient. For example, in some embodiments, the effective amount for one adult (body weight of 60 kg) is 1 mg to 1000 mg, for example, 25 mg to 600 mg, for example, 50 mg to 550 mg, for example, 100 mg to 500 mg of the DAAO inhibitor (for example, Compound (A)) per administration.

In some embodiments, the oral solid preparation is suitable for administering a high dose of a DAAO inhibitor (for example, Compound (A)) to the mammal. In some embodiments, the solid preparation of the present disclosure is administered to the mammal once daily.

The size of an oral solid preparation of the present disclosure may vary depending on the shape of the oral solid preparation (e.g., round shape, caplet shape, oblong shape, or the like). In some embodiments, the size is suited to patient administration.

Non-limiting examples of the oral solid preparation of the present disclosure include:
a tablet comprising 10 mg to 1000 mg of Compound (A) per tablet; and
a tablet comprising 10, 25, 50, 100, 125, 200, 250, 300, 400, 500, or 600 mg of Compound (A) per tablet.

### Combinations

An oral solid preparation of the present disclosure can be used in combination with one or more other types of drugs, which may be referred to as a “combination drug” hereinafter.

Combination drugs include other drugs and/or D-serine (D-SER) used to treat the disease or condition. In some embodiments, combination drugs may be chosen from the following.

(i) Antidepressants such as, for example, amitriptyline, amoxapine, bupropion, citalopram, clomipramine, desipramine, doxepin, duloxetine, elzasonan, escitalopram, fluvoxamine, fluoxetine, gepirone, imipramine, ipsapirone, maprotiline, nortriptyline, nefazodone, paroxetine, phenelzine, protriptyline, reboxetine, robaizotan, sertraline, sibutramine, thionisoxetine, tranylcypromine, trazodone, trimipramine, venlafaxine, equivalents thereof, pharmaceutically active isomers (singular or plural) and/or metabolites (singular or plural), and the like;

(ii) Atypical antipsychotics such as, for example, quetiapine and pharmaceutically active isomers (singular or plural) and/or metabolites thereof (singular or plural);

(iii) Antipsychotics such as, for example, amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone, equivalents thereof, pharmaceutically active isomers (singular or plural) and/or metabolites (singular or plural), and the like;

(iv) Anti-anxiety drugs such as, for example, alnespirone, azapirones, benzodiazepines, barbiturates, equivalents thereof, pharmaceutically active isomers (singular or plural) and/or metabolites (singular or plural), and the like. Examples of anti-anxiety drugs include adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam, equivalents thereof, pharmaceutically active isomers (singular or plural) and/or metabolites (singular or plural);

(v) Anticonvulsants such as, for example, carbamazepine, valproate, lamotrigine, and gabapentin, equivalents thereof, pharmaceutically active isomers (singular or plural) and/or metabolites (singular or plural), and the like;

(vi) Alzheimer's therapeutic agents such as, for example, donepezil, memantine, tacrine, equivalents thereof, pharmaceutically active isomers (singular or plural) and/or metabolites (singular or plural), and the like;

(vii) Parkinson's therapeutic agents such as, for example, deprenyl, L-dopa, Requip, and Mirapex, B-type monoamine oxidase (MAO-B) inhibitors such as, for example, selegiline and rasagiline, catecol-O-methyltransferase (COMT) inhibitors such as, for example, Tasmar, A-2 inhibitors, dopamine reuptake inhibitors, NMDA antagonists, nicotine agonists, dopamine agonists, inhibitors of neuronal nitric oxide synthase, equivalents thereof, pharmaceutically active isomers (singular or plural) and/or metabolites (singular or plural), and the like; (viii) Drugs for treating migraines, such as, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, equivalents thereof, pharmaceutically active isomers (singular or plural) and/or metabolites (singular or plural), and the like;

(ix) Cerebral infarction therapeutic agents such as, for example, abciximab, activase, NXY-059, citicoline, crobenetine, desmoteplase, repinotan, traxoprodil, equivalents thereof, pharmaceutically active isomers (singular or plural) and/or metabolites (singular or plural), and the like;

(x) Urinary incontinence therapeutic agents such as, for example, darafenacin, flavoxate, oxybutynin, propiverine, robalzotan, solifenacin, tolterodine, equivalents thereof, pharmaceutically active isomers (singular or plural) and/or metabolites (singular or plural), and the like;

(xi) Neuropathic pain therapeutic agents such as, for example, gabapentin, lidoderm, pregabalin, equivalents thereof, pharmaceutically active isomers (singular or plural) and/or metabolites (singular or plural), and the like;

(xii) Nociceptive pain therapeutic agents such as, for example, celecoxib, etoricoxib, lumiracoxib, rofecoxib, valdecoxib, diclofenac, loxoprofen, naproxen, paracetamol, equivalents thereof, pharmaceutically active isomers (singular or plural) and/or metabolites (singular or plural), and the like;

(xiii) Insomnia therapeutic agents such as, for example, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentabarbital, phenobarbital, propofol, roletamide, triclofos, secobarbital, zaleplon, Zolpidem, equivalents thereof, pharmaceutically active isomers (singular or plural) and/or metabolites (singular or plural), and the like; (xiv) Mood stabilizers such as, for example, carbamazepine, divalproex, gabapentin, lamotrigine, lithium, olanzapine, quetiapine, valproate, valproic acid, verapamil, equivalents thereof, pharmaceutically active isomers (singular or plural) and/or metabolites (singular or plural), and the like;

(xv) 5HT1B ligands, such as, for example, the compounds disclosed in WO 99/05134, WO 02/08212, and the like;

(xvi) mGluR2 agonist;

(xvii) Alpha 7 nicotine agonists, such as, for example, the compounds disclosed in WO 96/006098, WO 97/030998, WO 99/003859, WO 00/042044, WO 01/029034, WO 01/60821, WO 01/36417, WO 02/096912, WO 03/087102, WO 03/087103, WO 03/087104, WO 2004/016617, WO 2004/016616, and WO 2004/019947;

(xviii) Chemokine receptor CCR1 inhibitors; and

(xix) Delta opioid agonists, such as, for example, the compounds disclosed in WO 97/23466 and WO 02/094794, and the like.

In some embodiments, an oral solid preparation of the present disclosure can be used in combination with other drugs used to prevent or treat ataxic disorders. Non-limiting examples of drugs to prevent or treat ataxic disorders include D-serine, D-serine ethyl ester, D-cycloserine, amantazine or amantazine hydrochloride (“Symmetrel”), buspirone (“Buspar”), acetazolamide (“Diamox”), topiramate (“Topamax”), divalproex sodium (“Depakote)”), L-dopa (“Sinemet”), proplanolol (“Inderal”), primidone (“Mysoline”), clonazepam (“Klonopin”), levetiracetam (“Keppra”), carbamazepine (“Tegretol”), gabapentine (“Neurontin”), baclofen (“Lioresal”), ondansetron (“Zofran”), tizanidine (“Zanaflex”), and pramipexole (“Mirapex”).

When an oral solid preparation of the present disclosure is used in combination with a combination drug, the oral solid preparation and the combination drug may be administered at the same time or at different times, in the same preparation or in different preparations.

In some embodiments, an oral solid preparation of the present disclosure and a combination drug may be administered to the mammal as separate preparations or as a single preparation comprising the oral solid preparation and the combination drug.

The dosage amount of the combination drug may be selected based on the clinically used dose of the combination drug. Moreover, the compounding ratio of an oral solid preparation of the present disclosure and the combination drug can be selected based on the administration target (i.e., mammal), route of administration, disease, symptom, combination, and the like. For example, when the mammal is a human, 0.01 to 100 parts by weight of the combination drug may be used with respect to 1 part by weight of the oral solid preparation.

In some embodiments, enhanced clinical effects may be obtained when administering a combination therapy (i.e., an oral solid preparation disclosed herein and a combination drug), such as: (1) an effect of enhancing the action of the DAAO inhibitor or the combination drug (i.e., a synergistic effect); (2) an effect of reducing the dosage amount of the DAAO inhibitor or the combination drug (i.e., an effect of reducing the dosage amount when compared to administering each drug alone); and/or (3) an effect of reducing a secondary action of the DAAO inhibitor or the combination drug.

### EXAMPLES

Some embodiments of the present disclosure are further described below through examples, comparative examples, and test examples. The examples are intended to be illustrative and are not meant in any way to limit the scope of the disclosure. Accordingly, the present disclosure is not limited to the specific embodiments described through examples, comparative examples, and test examples herein.

Products conforming to the 17^{th} Edition Japanese Pharmacopoeia, pharmaceutical standards outside of Japanese Pharmacopoeia, or standards of medical package inserts 2003 were used as preparation additives in the following examples, comparative examples, and test examples.

### Comparative Example 1

Hydroxypropyl cellulose (13.5 g, grade L, produced by NIPPON SODA CO., LTD.) was dissolved in purified water (211.5 g) to prepare a binder solution. Compound (A) (150 g), D-mannitol (214.5 g, PEARLITOL 50C, produced by Roquette Co.), and microcrystalline cellulose (45 g, CEOLUS PH-101, produced by Asahi Kasei Corporation) were granulated in a fluid-bed granulator (LAB-1, manufactured by Powrex Co.) while spraying a binder solution and then dried to obtain a granulated powder. The granulated powder was then milled, 282.0 g of the obtained milled powder was weighed, and sodium starch glycolate (15.0 g, type A. Primojel (registered trademark), produced by DFE Pharma) and magnesium stearate (produced by DFE Pharma) (3.0 g, produced by Taihei Chemical Industrial Co., Ltd.) were added and mixed to obtain a mixed powder. The obtained mixed powder was made into tablets using a rotary tablet press (VELA5, manufactured by KIKUSUI SEISAKUSHO LTD.) to obtain uncoated tablets having a weight of 300 mg per tablet and a diameter of 9 mm. All of the obtained uncoated tablets were charged into a Doria coater (DRC-200, manufactured by Powrex Co.), and an aqueous dispersion of hypromellose, Macrogol 6000, titanium dioxide, red ferric oxide, and yellow ferric oxide was sprayed to produce 12.2 mg of a film coating per tablet and obtain Preparation 1 (film-coated tablets). The composition of Preparation 1 per tablet is shown in Table 1.

TABLE 1
Composition of Preparation 1 per Tablet

| Component | Amount (mg) |
|---|---|
| Compound (A) | 100 |
| D-mannitol (PEARLITOL 50C) | 143 |
| Microcrystalline Cellulose (CEOLUS PH-101) | 30 |
| Hydroxypropyl Cellulose (HPC-L) | 9 |
| Sodium Starch Glycolate Type A | 15 |
| Magnesium Stearate | 3 |
| Hypromellose | 9 |
| Macrogol 6000 | 9 |
| Titanium Dioxide | 1 |
| Red Ferric Oxide | 0.1 |
| Yellow Ferric Oxide | 0.1 |
| Total | 312.2 |

### Example 1

Hydroxypropyl cellulose (13.5 g, grade L, produced by NIPPON SODA CO., LTD.) was dissolved in purified water (211.5 g) to prepare a binder solution. Compound (A) (150 g), D-mannitol (192 g, PEARLITOL 50C, produced by Roquette Co.), and microcrystalline cellulose (45 g, CEOLUS PH-101, produced by Asahi Kasei Corporation) were granulated in a fluid-bed granulator (LAB-1, manufactured by Powrex Co.) while spraying a binder solution and then dried to obtain a granulated powder. The granulated powder was then milled, 213.6 g of the obtained milled powder was weighed, and low-substituted hydroxypropyl cellulose (24.0 g, grade LH-21, produced by Shin-Etsu Chemical Co., Ltd.) (containing 11% hydroxypropoxy group (dry weight)), and magnesium stearate (2.40 g, produced by Taihei Chemical Industrial Co., Ltd.) were added and mixed to obtain a mixed powder. The obtained mixed powder was made into tablets using a rotary tablet press (VELA5, manufactured by KIKUSUI SEISAKUSHO LTD.) to obtain uncoated tablets having a weight of 300 mg per tablet and a diameter of 9 mm. All of the obtained uncoated tablets were inserted into a Doria coater (DRC-200, manufactured by Powrex Co.), and an aqueous dispersion of hypromellose, Macrogol 6000, titanium oxide, red ferric oxide, and yellow ferric oxide was sprayed to have 12.2 mg of a film coating per tablet to obtain Preparation 2 (film-coated tablets). The composition of Preparation 2 per tablet is shown in Table 2.

TABLE 2
Composition of Preparation 2 per Tablet

| Component | Amount (mg) |
|---|---|
| Compound (A) | 100 |
| D-mannitol (PEARLITOL 50C) | 128 |
| Microcrystalline Cellulose (CEOLUS PH-101) | 30 |
| Hydroxypropyl Cellulose (HPC-L) | 9 |
| Low-substituted Hydroxypropyl Cellulose (LH-21) | 30 |
| Magnesium Stearate | 3 |
| Hypromellose | 9 |
| Macrogol 6000 | 2 |
| Titanium Dioxide | 1 |
| Red Ferric Oxide | 0.1 |
| Yellow Ferric Oxide | 0.1 |
| Total | 312.2 |

### Example 2

Preparation 3 (film-coated tablets), that contains 100 mg of Compound (A) per tablet, can be produced by a similar production method to the one described in Example 1. The composition of Preparation 3 per tablet is shown in Table 3.

TABLE 3
Composition of Preparation 3 per Tablet

| Component | Amount (mg) |
|---|---|
| Compound (A) | 100 |
| D-mannitol (PEARLITOL 50C) | 128 |
| Microcrystalline Cellulose (CEOLUS PH-101) | 30 |
| Hydroxypropyl Cellulose (HPC-L) | 9 |
| Low-substituted Hydroxypropyl Cellulose (LH-21) | 30 |
| Magnesium Stearate | 3 |
| Hypromellose (TC-5R) | 9 |
| Titanium Dioxide | 1 |
| Red Ferric Oxide | 0.1 |
| Yellow Ferric Oxide | 0.1 |
| Total | 310.2 |

### Example 3

Compound (A) (1 g), D-mannitol (0.52 g, PEARLITOL 50C, manufactured by Roquette Co.), microcrystalline cellulose (0.2 g, CEOLUS PH-101, produced by Asahi Kasei Corporation), low-substituted hydroxypropyl cellulose (0.1 g, grade LH-21, produced by Shin-Etsu Chemical Industry Co., Ltd.), and hydroxypropyl cellulose (0.06 g, grade L, produced by Nippon Soda Co., Ltd.) were weighed and placed in a mortar. The components were granulated after adding purified water, and the obtained wet powder was dried in a vacuum dryer (DP-33, manufactured by Yamato Scientific Co., Ltd.) to obtain a granulated powder. The granulated powder was then milled, and low-substituted hydroxypropyl cellulose (0.1 g, grade LH-21, produced by Shin-Etsu Chemical Co., Ltd.) and magnesium stearate (0.02 g, produced by Taihei Chemical Industrial Co., Ltd.) were added and mixed into the obtained milled powder to obtain a mixed powder. The obtained mixed powder was made into tablets using a tabletop tablet press (HANDTAB-100, manufactured by Ichihashi Seiki) at a compression force of approximately 7 kN to obtain Preparation 4 (uncoated tablets) having a weight of 200 mg per tablet and a diameter of 9 mm. The composition of Preparation 4 per tablet is shown in Table 4.

TABLE 4
Composition of Preparation 4 per Tablet

| Component | Amount (mg) |
|---|---|
| Compound (A) | 100 |
| D-mannitol (PEARLITOL 50C) | 52 |
| Microcrystalline Cellulose (CEOLUS PH-101) | 20 |
| Low-substituted Hydroxypropyl Cellulose (LH-21) | 20 |
| Hydroxypropyl Cellulose (HPC-L) | 6 |
| Magnesium Stearate | 2 |
| Total | 200 |

### Example 4

Compound (A) (165.0 g), D-mannitol (85.80 g, PEARLITOL 50C, manufactured by Roquette Co.), microcrystalline cellulose (33 g, CEOLUS PH-101, produced by Asahi Kasei Corporation), low-substituted hydroxypropyl cellulose (16.5 g, grade LH-21, produced by Shin-Etsu Chemical Industry Co., Ltd.), and hydroxypropyl cellulose (9.9 g, grade L, produced by Nippon Soda Co., Ltd.) were weighed and placed in a high-shear granulator (FM-VG-01, Powrex Co.) and granulated by adding purified water. The obtained wet powder was wet-milled and dried in a fluid-bed dryer (LAB-1, Powrex Co.) to obtain a granulated powder. The granulated powder was then milled, 270.7 g of the obtained milled powder was weighed, and low-substituted hydroxypropyl cellulose (14.4 g, grade LH-21, produced by Shin-Etsu Chemical Co., Ltd.) and magnesium stearate (2.88 g, produced by Taihei Chemical Industrial Co., Ltd.) were added and mixed to obtain a mixed powder. The obtained mixed powder was made into tablets using a rotary tablet press (VELA5, manufactured by KIKUSUI SEISAKUSHO LTD.) at a compression force of 15 to 17 kN, and uncoated tablets were obtained having a weight of 600 mg per tablet, a length of 14 mm, and a breadth of 8 mm. All of the obtained uncoated tablets were charged into a Doria coater (DRC-200, manufactured by Powrex Co.), and an aqueous dispersion of hypromellose (TC-5R, produced by Shin-Etsu Chemical Co., Ltd.), titanium dioxide (A-HR, produced by FREUND), red ferric oxide, and yellow ferric oxide (both produced by VENATOR PIGMENTS) was sprayed to have 20.4 mg of a film coating per tablet to obtain Preparation 5 (film-coated tablets). The composition of Preparation 5 per tablet is shown in Table 5.

In relation to other doses (comprising 25 mg or 100 mg of Compound (A) per tablet), it is also possible to manufacture by using the same production method while having the same ratio of additives except for D-mannitol.

TABLE 5
Composition of Preparation 5 per Tablet

| Component | Amount (mg) |
|---|---|
| Compound (A) | 300 |
| D-mannitol (PEARLITOL 50C) | 156 |
| Microcrystalline Cellulose (CEOLUS PH-101) | 60 |
| Low-substituted Hydroxypropyl Cellulose (LH-21) | 60 |
| Hydroxypropyl Cellulose (HPC-L) | 18 |
| Magnesium Stearate | 6 |
| Hypromellose (TC-5R) | 18 |
| Titanium Dioxide | 2 |
| Red Ferric Oxide | 0.2 |
| Yellow Ferric Oxide | 0.2 |
| Total | 620.4 |

### Example 5

Preparation 6 (film-coated tablets), which contains 500 mg of Compound (A) per tablet, can be produced using a similar production method to Example 4. The composition of Preparation 6 per tablet is shown in Table 6.

TABLE 6
Composition of Preparation 6 per Tablet

| Component | Amount (mg) |
|---|---|
| Compound (A) | 500 |
| D-mannitol (PEARLITOL 50C) | 260 |
| Microcrystalline Cellulose (CEOLUS PH-101) | 100 |
| Low-substituted Hydroxypropyl Cellulose (LH-21) | 100 |
| Hydroxypropyl Cellulose (HPC-L) | 30 |
| Magnesium Stearate | 10 |
| Hypromellose (TC-5R) | 30 |
| Titanium Dioxide | 3.3 |
| Red Ferric Oxide | 0.3 |
| Yellow Ferric Oxide | 0.3 |
| Total | 1033.9 |

### Example 6

Compound (A) (165 g), D-mannitol (85.8 g, PEARLITOL 50C, manufactured by Roquette Co.), microcrystalline cellulose (33 g, CEOLUS PH-101, produced by Asahi Kasei Corporation), low-substituted hydroxypropyl cellulose (16.5 g, grade LH-21, produced by Shin-Etsu Chemical Industry Co., Ltd.), and hydroxypropyl cellulose (9.9 g, grade L, produced by Nippon Soda Co., Ltd.) were weighed and placed in a high-shear granulator (FM-VG-01, Powrex Co.) and then granulated by adding purified water. The obtained wet powder was wet-milled and dried in a fluid-bed dryer (LAB-1, Powrex Co.) to obtain a granulated powder. The granulated powder was then milled, 263.2 g of the obtained milled powder was weighed, and low-substituted hydroxypropyl cellulose (14 g, grade LH-21, produced by Shin-Etsu Chemical Co., Ltd.) and magnesium stearate (2.8 g, produced by Taihei Chemical Industrial Co., Ltd.) were added and mixed to obtain a mixed powder. The obtained mixed powder was made into tablets using a rotary tablet press (VELA5, manufactured by KIKUSUI SEISAKUSHO LTD.) at a compression force of 25 kN, and uncoated tablets were obtained having weight of 1000 mg per tablet, a length of 17.5 mm, and a breadth of 9.5 mm. All of the obtained uncoated tablets were charged into a pan coater (HICOATER HC-LABO-20, manufactured by FREUND Corporation), and an aqueous dispersion of hypromellose (TC-5R, produced by Shin-Etsu Chemical Co., Ltd.), titanium dioxide (A-HR, produced by FREUND), red ferric oxide and yellow ferric oxide (both produced by VENATOR PIGMENTS) was sprayed to produce 33.9 mg of a film coating per tablet and obtain Preparation 7 (film-coated tablets). The composition of Preparation 7 per tablet is shown in Table 7.

TABLE 7
Composition of Preparation 7 per Tablet

| Component | Amount (mg) |
|---|---|
| Compound (A) | 500 |
| D-mannitol (PEARLITOL 50C) | 260 |
| Microcrystalline Cellulose (CEOLUS PH-101) | 100 |
| Low-substituted Hydroxypropyl Cellulose (LH-21) | 100 |
| Hydroxypropyl Cellulose (HPC-L) | 30 |
| Magnesium Stearate | 10 |
| Hypromellose (TC-5R) | 30 |
| Titanium Dioxide | 3.3 |
| Red Ferric Oxide | 0.3 |
| Yellow Ferric Oxide | 0.3 |
| Total | 1033.9 |

### Example 7

Compound (A) (195 g), D-mannitol (53.98 g, PEARLITOL 50C, manufactured by Roquette Co.), microcrystalline cellulose (32.76 g, CEOLUS PH-101, produced by Asahi Kasei Corporation), low-substituted hydroxypropyl cellulose (16.38 g, grade LH-21, produced by Shin-Etsu Chemical Industry Co., Ltd.) and hydroxypropyl cellulose (9.828 g, grade L, produced by Nippon Soda Co., Ltd.) were weighed and placed in a high-shear granulator (FM-VG-01, Powrex Co.) and granulated by adding purified water. The obtained wet powder was wet-milled and dried in a fluid-bed dryer (LAB-1, Powrex Co.) to obtain a granulated powder. The granulated powder was then milled, 268.5 g of the obtained milled powder was weighed, and low-substituted hydroxypropyl cellulose (14.28 g, grade LH-21, produced by Shin-Etsu Chemical Co., Ltd.) and magnesium stearate (2.856 g, produced by Taihei Chemical Industrial Co., Ltd.) were added and mixed to obtain a mixed powder. The obtained mixed powder was made into tablets using a rotary tablet press (VELA5, manufactured by KIKUSUI SEISAKUSHO LTD.) at a compression force of 20 kN, and uncoated tablets were obtained having weight of 840 mg per tablet, a length of 16 mm, and a breadth of 9 mm. All of the obtained uncoated tablets were charged into a pan coater (HICOATER HC-LABO-20, manufactured by FREUND Corporation), and an aqueous dispersion of hypromellose (TC-5R, produced by Shin-Etsu Chemical Co., Ltd.), titanium dioxide (A-HR, produced by FREUND), red ferric oxide and yellow ferric oxide (both produced by VENATOR PIGMENTS) was sprayed to create 28.6 mg of a film coating per tablet and obtain Preparation 8 (film-coated tablets). The composition of Preparation 8 per tablet is shown in Table 8.

TABLE 8
Composition of Preparation 8 per Tablet

| Component | Amount (mg) |
|---|---|
| Compound (A) | 500 |
| D-mannitol (PEARLITOL 50C) | 138.4 |
| Microcrystalline Cellulose (CEOLUS PH-101) | 84 |
| Low-substituted Hydroxypropyl Cellulose (LH-21) | 84 |
| Hydroxypropyl Cellulose (HPC-L) | 25.2 |
| Magnesium Stearate | 8.4 |
| Hypromellose (TC-5R) | 25.2 |
| Titanium Dioxide | 2.8 |
| Red Ferric Oxide | 0.3 |
| Yellow Ferric Oxide | 0.3 |
| Total | 868.6 |

### Example 8

Hydroxypropyl cellulose (810 g, grade L, produced by NIPPON SODA CO., LTD.) was dissolved in purified water (12690 g) to prepare a binder solution. Compound (A) (900 g), D-mannitol (19620 g, PEARLITOL 50C, produced by Roquette Co.), and microcrystalline cellulose (2700 g, CEOLUS PH-101, produced by Asahi Kasei Corporation) were granulated in a fluid-bed granulator (FD-WGS-30, manufactured by Powrex Co.) while spraying a binder solution and then dried to obtain a granulated powder. The granulated powder was manufactured for 2 batches. The granulated powder was then milled. 44060 g of the obtained milled powder was weighed, and low-substituted hydroxypropyl cellulose (4950 g, grade LH-21, produced by Shin-Etsu Chemical Co., Ltd.) (containing 11% hydroxypropoxy group (dry weight)), and magnesium stearate (495 g, produced by Taihei Chemical Industrial Co., Ltd.) were added and mixed to obtain a mixed powder. The obtained mixed powder was made into tablets using a rotary tablet press (AQUARIUS 08242L2JI, manufactured by KIKUSUI SEISAKUSHO LTD.) to obtain uncoated tablets having a weight of 300 mg per tablet and a diameter of 9 mm. 40500 g of the obtained uncoated tablets were inserted into a Doria coater (DRC-900S, manufactured by Powrex Co.), and an aqueous dispersion of hypromellose, Macrogol 6000, titanium oxide, red ferric oxide, and yellow ferric oxide was sprayed to create 12.2 mg of a film coating per tablet and obtain Preparation 9 (film-coated tablets). The composition of Preparation 9 per tablet is shown in Table 9.

TABLE 9
Composition of Preparation 9 per Tablet

| Component | Amount (mg) |
|---|---|
| Compound (A) | 10 |
| D-mannitol (PEARLITOL 50C) | 218 |
| Microcrystalline Cellulose (CEOLUS PH-101) | 30 |
| Hydroxypropyl Cellulose (HPC-L) | 9 |
| Low-substituted Hydroxypropyl Cellulose (LH-21) | 30 |
| Magnesium Stearate | 3 |
| Hypromellose (TC-5R) | 9 |
| Titanium Dioxide | 1 |
| Red Ferric Oxide | 0.1 |
| Yellow Ferric Oxide | 0.1 |
| Total | 310.2 |

### Example 9

Hydroxypropyl cellulose (810 g, grade L, produced by NIPPON SODA CO., LTD.) was dissolved in purified water (12690 g) to prepare a binder solution. Compound (A) (2250 g), D-mannitol (18270 g, PEARLITOL 50C, produced by Roquette Co.), and microcrystalline cellulose (2700 g, CEOLUS PH-101, produced by Asahi Kasei Corporation) were granulated in a fluid-bed granulator (FD-WGS-30, manufactured by Powrex Co.) while spraying a binder solution and then dried to obtain a granulated powder. The granulated powder was manufactured for 2 batches. The granulated powder was then milled. 44060 g of the obtained milled powder was weighed, and low-substituted hydroxypropyl cellulose (4950 g, grade LH-21, produced by Shin-Etsu Chemical Co., Ltd.) (containing 11% hydroxypropoxy group (dry weight)), and magnesium stearate (495 g, produced by Taihei Chemical Industrial Co., Ltd.) were added and mixed to obtain a mixed powder. The obtained mixed powder was made into tablets using a rotary tablet press (AQUARIUS 08242L2JI, manufactured by KIKUSUI SEISAKUSHO LTD.) to obtain uncoated tablets having a weight of 300 mg per tablet and a diameter of 9 mm. 40500 g of the obtained uncoated tablets were inserted into a Doria coater (DRC-900S, manufactured by Powrex Co.), and an aqueous dispersion of hypromellose, Macrogol 6000, titanium oxide, red ferric oxide, and yellow ferric oxide was sprayed to create 12.2 mg of a film coating per tablet and obtain Preparation 10 (film-coated tablets). The composition of Preparation 10 per tablet is shown in Table 10.

TABLE 10
Composition of Preparation 10 per Tablet

| Component | Amount (mg) |
|---|---|
| Compound (A) | 25 |
| D-mannitol (PEARLITOL 50C) | 203 |
| Microcrystalline Cellulose (CEOLUS PH-101) | 30 |
| Hydroxypropyl Cellulose (HPC-L) | 9 |
| Low-substituted Hydroxypropyl Cellulose (LH-21) | 30 |
| Magnesium Stearate | 3 |
| Hypromellose (TC-5R) | 9 |
| Titanium Dioxide | 1 |
| Red Ferric Oxide | 0.1 |
| Yellow Ferric Oxide | 0.1 |
| Total | 310.2 |

### Example 10

Preparations 11-13 can be prepared using similar production methods to those described above. The compositions of Preparations 11-13 (on a per tablet basis) are shown in Tables 11-13, respectively.

TABLE 11
Composition of Preparation 11 per Tablet

| Component | | Amount (mg) |
|---|---|---|
| Core | Compound (A) | 250 |
| Tablet | D-mannitol (PEARLITOL 50C) | 206 |
| | Microcrystalline Cellulose (CEOLUS PH-101) | 60 |
| | Low-substituted Hydroxypropyl Cellulose | 60 |
| | (LH-21) | |
| | Hydroxypropyl Cellulose (HPC-L) | 18 |
| | Magnesium Stearate | 6 |
| Coating | Hypromellose (TC-5R) | 8.6 |
| Material | Titanium Dioxide | 3.3 |
| | Hydroxypropyl Cellulose | 8.6 |
| | Total | 620.5 |

TABLE 12
Composition of Preparation 12 per Tablet

| Component | | Amount (mg) |
|---|---|---|
| Core | Compound (A) | 125 |
| Tablet | D-mannitol (PEARLITOL 50C) | 331 |
| | Microcrystalline Cellulose (CEOLUS PH-101) | 60 |
| | Low-substituted Hydroxypropyl Cellulose | 60 |
| | (LH-21) | |
| | Hydroxypropyl Cellulose (HPC-L) | 18 |
| | Magnesium Stearate | 6 |
| Coating | Hypromellose (TC-5R) | 8.6 |
| Material | Titanium Dioxide | 3.3 |
| | Hydroxypropyl Cellulose | 8.6 |
| | Total | 620.5 |

TABLE 13
Composition of Preparation 13 per Tablet

| Component | Amount (mg) |
|---|---|
| Compound (A) | 50 |
| D-mannitol (PEARLITOL 50C) | 178 |
| Microcrystalline Cellulose (CEOLUS PH-101) | 30 |
| Hydroxypropyl Cellulose (HPC-L) | 9 |
| Low-substituted Hydroxypropyl Cellulose (LH-21) | 30 |
| Magnesium Stearate | 3 |
| Hypromellose (TC-5R) | 9 |
| Titanium Dioxide | 1 |
| Red Ferric Oxide | 0.1 |
| Yellow Ferric Oxide | 0.1 |
| Total | 310.2 |

### Test Example 1: Comparison of Dissolution of the Preparations Obtained in Comparative Example 1 and Example 1

The dissolution properties of preparations obtained in Comparative Example 1 and Example 1 were compared using a dissolution test device (manufactured by Toyama Sangyo Co., Ltd.). Dissolution properties were compared for unaged preparations and preparations placed in a glass bottle without a cap and stored for two weeks at a temperature of 40° C. and a relative humidity of 900%. Following the dissolution test method (paddle method) described in the Japanese Pharmacopoeia, testing was performed at 50 revolutions per minute using 900 mL of a 0.05 mol/L phosphate buffer (pH 6.8) containing 0.1% sodium dodecyl sulfate (SDS) as the test solution. The test solution was collected 5 minutes, 10 minutes, 15 minutes, 20 minutes, 30 minutes, 45 minutes, and 60 minutes after the start of the test, and the dissolution rate of Compound (A) was measured using a high performance liquid chromatograph (manufactured by Waters Corporation). The results are shown in Table 14. The values in the table indicate the average value of the dissolution rates of 6 film-coated tablets.

TABLE 14

| | Comparative Example 1 | | Example 1 | |
|---|---|---|---|---|
| | | Product Stored | | Product Stored |
| Time of | | at 40° C./90% RH | | at 40° C./90% RH |
| Sampling | Unaged | for Two Weeks | Unaged | for Two Weeks |
| 5 minutes | 30% | 19% | 35% | 45% |
| 10 minutes | 70% | 38% | 84% | 72% |
| 15 minutes | 85% | 48% | 93% | 83% |
| 20 minutes | 91% | 56% | 96% | 88% |
| 30 minutes | 95% | 69% | 96% | 92% |
| 45 minutes | 96% | 78% | 98% | 95% |
| 60 minutes | 96% | 84% | 97% | 96% |

The initial dissolution rates for the preparations obtained in Comparative Example 1 and Example 1 were similar, with 95% at 30 minutes and 96% at 60 minutes for the preparation of Comparative Example 1, and 96% at 30 minutes and 97% at 60 minutes for the preparation of Example 1. However, a dissolution delay was confirmed after storage for the preparation of Comparative Example 1, with the dissolution rate of the products, having been stored for 2 weeks at a temperature of 40° C. and a relative humidity of 90%, being 69% at 30 minutes and 84% at 60 minutes for the preparation in Comparative Example 1, and 92% at 30 minutes and 96% in 60 minutes for the preparation in Example 1. Additionally, it was observed that in the preparation of the present disclosure containing L-HPC, 92% of Compound (A) was dissolved within 30 minutes, and the preparation exhibited improved dissolution stability after storage.

### Test Example 2: Measuring Dissolution of the Preparation Obtained in Example 4

The dissolution properties of Preparation 5 obtained in Example 4 were measured using a dissolution test device (manufactured by Toyama Sangyo Co., Ltd.) for unaged samples and samples placed into a glass bottle without a cap stored for two weeks at a temperature of 40° C. and a relative humidity of 90%. Following the dissolution test method (paddle method) described in the Japanese Pharmacopoeia, testing was performed at 50 revolutions per minute using 900 mL of a 0.05 mol/L phosphate buffer (pH 6.8) as the test solution. The test solution was collected 5 minutes, 10 minutes, 15 minutes, 30 minutes, 45 minutes, and 60 minutes after the start of the test, and the dissolution rate of Compound (A) was measured using a high performance liquid chromatograph (manufactured by Waters Corporation). The results are shown in Table 15. The values in the table indicate the average value of the dissolution rates of 3 film-coated tablets.

TABLE 15

Example 4

| | | Product Stored |
|---|---|---|
| Time of | | at 40° C./90% RH |
| Sampling | Initial | for Two Weeks |
| 5 minutes | 6% | 18% |
| 10 minutes | 45% | 52% |
| 15 minutes | 73% | 64% |
| 30 minutes | 90% | 78% |
| 45 minutes | 96% | 85% |
| 60 minutes | 99% | 90% |

Thus, 78% of Compound (A) was dissolved within 30 minutes for the preparation obtained from Example 4, which contains L-HPC, indicating excellent dissolution stability after storage.

### Test Example 3: Comparison of Dissolution of the Preparations Obtained in Example 6 and Example 7

The dissolution properties of preparations obtained in Example 6 and Example 7 were measured using a dissolution test device VK7010 (manufactured by Agilent) for unaged samples and samples placed into a glass bottle without a cap and stored for two weeks at a temperature of 40° C. and a relative humidity of 90%. Following the dissolution test method (paddle method) described in the Japanese Pharmacopoeia, testing was performed at 50 revolutions per minute using 900 mL of a 0.05 mol/L phosphate buffer (pH 6.8) containing 0.2% of cetyltrimethylammonium bromide (CTAB) as the test solution. The dissolution rate of Compound (A) was measured at 5 minutes, 10 minutes, 15 minutes, 30 minutes, 45 minutes, and 60 minutes after the start of the test. The values in Table 16 indicate the average value of the dissolution rates of 3 film-coated tablets.

TABLE 16

| | Example 6 | | Example 7 | |
|---|---|---|---|---|
| | (Preparation 7) | | (Preparation 8) | |
| | | Product Stored | | Product Stored |
| Time of | | at 40° C./90% RH | | at 40° C./90% RH |
| Sampling | Initial | for Two Weeks | Initial | for Two Weeks |
| 5 minutes | 0% | 3% | 0% | 1% |
| 10 minutes | 16% | 27% | 17% | 15% |
| 15 minutes | 39% | 49% | 42% | 34% |
| 30 minutes | 89% | 83% | 87% | 76% |
| 45 minutes | 97% | 92% | 94% | 86% |
| 60 minutes | 99% | 96% | 96% | 90% |

Thus, 83% of Compound (A) in Preparation 7 dissolved within 30 minutes, and 76% of Compound (A) in Preparation 8 dissolved within 30 minutes, indicating that both preparations have excellent dissolution stability after storage.

Claims or descriptions that include “or” or “and/or” between at least one members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Furthermore, the disclosure encompasses all variations, combinations, and permutations in which at least one limitation, element, clause, and descriptive term from at least one of the listed claims is introduced into another claim. For example, any claim that is dependent on another claim can be modified to include at least one limitation found in any other claim that is dependent on the same base claim. Where elements are presented as lists, such as, e.g., in Markush group format, each subgroup of the elements is also disclosed, and any element(s) can be removed from the group. It should be understood that, in general, where the disclosure, or aspects of the disclosure, is/are referred to as comprising particular elements and/or features, embodiments of the disclosure or aspects of the disclosure consist, or consist essentially of, such elements and/or features. For purposes of simplicity, those embodiments have not been specifically set forth in haec verba herein. Where ranges are given (such as, e.g., from [X] to [Y]), endpoints (such as, e.g., [X] and [Y] in the phrase “from [X] to [Y]”) are included unless otherwise indicated. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those of ordinary skill in the art will recognize or be able to ascertain using no more than routine experimentation, many equivalents to the specific embodiments of the disclosure described herein. Such equivalents are intended to be encompassed by the following claims.

1. An oral solid preparation comprising a D-amino acid oxidase (DAAO) inhibitor, a low-substituted hydroxypropyl cellulose (L-HPC), and an additive, wherein the DAAO inhibitor is a pyridazinone derivative.

2. The oral solid preparation according to claim 1, wherein the oral solid preparation is a tablet.

3. The oral solid preparation according to claim 1, wherein the additive is chosen from fillers, binders, disintegrants, lubricants, and combinations of any of the foregoing.

4. The oral solid preparation according to claim 1, wherein the DAAO inhibitor is chosen from compounds of Formula (I): 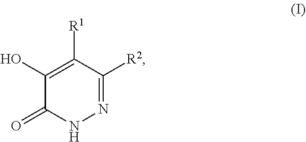
R¹ is hydrogen, fluorine, or trifluoromethyl;
R² is chosen from -XYR³ groups;
X and Y are independently chosen from a bond, oxygen, —C(O), —S(O)ₙ groups, —C(O)NR⁴ groups, —S(O)₂NR⁴ groups, —NR⁴ groups, 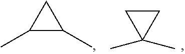
and —CR⁴R⁵— groups, wherein:
X and Y are not both a bond; and
when neither X nor Y is a bond, then at least one of X and Y is chosen from —CR⁴R⁵— groups;
n is 0, 1, or 2;
each R⁴ is independently chosen from hydrogen, C₁-C₆ alkyl groups, and C₁-C₆ haloalkyl groups;
each R⁵ is independently chosen from hydrogen, C₁-C₆ alkyl groups, C₁-C₆ haloalkyl groups, and ═CH—;
R³ is chosen from saturated or unsaturated carbocyclic or heterocyclic ring systems of 3 to 10 members, wherein the ring system is optionally substituted by at least one substituent chosen from halogen groups, hydroxyl, cyano, oxo, C₁-C₆alkyl groups, C₂-C₆ alkenyl groups, C₁-C₆ haloalkyl groups, C₁-C₆ hydroxyalkyl groups, C₁-C₆ alkoxy groups, C₁-C₆ haloalkoxy groups, C₁-C₆ alkylthiogroups, C₁-C₆ alkylsulfinyl groups, C₁-C₆ alkylsulfonyl groups, C₁-C₆ alkylcarbonyl groups, C₁-C₆ alkylcarbonyloxy groups, C₁-C₆ alkoxycarbonyl groups, amino, —CON(R⁶)₂ groups, C₁-C₆ alkyl amino groups, di-(C₁-C₆ alkyl) amino groups, C₃-C₆ cycloalkyl groups, C₃-C₆ cycloalkyloxy groups, C₃-C₆ cycloalkyl methyl groups, —[O]p-(CH₂)q-O—R⁷ groups, and saturated or unsaturated heterocyclic rings of 4 to 6 members optionally substituted by at least one substituent chosen from C₁-C₄alkyl groups and C₁-C₄alkoxy groups;
each R⁶ is independently chosen from hydrogen and C₁-C₆ alkyl groups;
p is 0 or 1;
q is 1,2,3, or 4; and
R⁷ is chosen from C₁-C₆ alkyl groups,
and pharmaceutically acceptable salts thereof.

5. The oral solid preparation according to claim 1, wherein the DAAO inhibitor is chosen from 4-hydroxy-6-{2-[4-(trifluoromethyl)phenyl]ethyl}pyridazine-3(2H)-one and pharmaceutically acceptable salts thereof.

6. The oral solid preparation according to claim 1, wherein the DAAO inhibitor is chosen from compounds of Formula (I)-a: 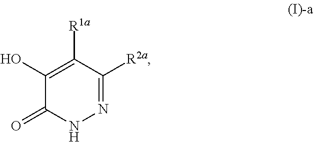
wherein:
R^{1a} is hydrogen, fluorine, or trifluoromethyl;
R^{2a} is chosen from C₂-C₆ alkyl groups, C₃-C₈ cycloalkyl groups, and tetrahydropyranyl, each of which may be optionally substituted with at least one substituent, or R^{2a} is chosen from —NR^{3a}R^{4a} groups;
R^{3a} and R^{4a} are independently chosen from hydrogen and C₁-C₆ alkyl groups, or R^{3a} and R^{4a} form a saturated or unsaturated heterocyclic ring of 4 to 8 members together with a bonded nitrogen atom, and each alkyl group or heterocyclic ring may be optionally substituted by at least one substituent; and
the optional substituents for R^{2a}, R^{3a}, and R^{4a} are independently chosen from halogen groups, hydroxyl, cyano, carboxyl, C₁-C₆ alkyl groups, difluoromethyl, trifluoromethyl, C₁-C₆ alkoxy groups, difluoromethoxy, and trifluoromethoxy, provided that the compound is not:
2,3-dihydro-4-hydroxy-6-morpholinopyridazine-3-one, or
6-amino-4-hydroxy-pyridazinone,
and pharmaceutically acceptable salts thereof.

7. The oral solid preparation according to claim 1, wherein the oral solid preparation comprises 10 mg to 1000 mg of the DAAO inhibitor per preparation unit.

8. The oral solid preparation according to claim 1, wherein one type of L-HPC is used.

9. The oral solid preparation according to claim 1, wherein two or more types of L-HPC is used.

10. The oral solid preparation according to claim 1, wherein the content of L-HPC is 1% to 20% by weight.

11. An oral solid preparation comprising:
3% to 60% by weight of 4-hydroxy-6-{2-[4-(trifluoromethyl)phenyl]ethyl}pyridazine-3(2H)-one;
3% to 15% by weight of a L-HPC;
15% to 85% by weight of a filler;
1% to 10% by weight of a binder; and
0.2% to 3% by weight of a lubricant.

12. The oral solid preparation according to claim 3, wherein the filler is chosen from mannitol, microcrystalline cellulose, starches, and combinations of any of the foregoing.

13. The oral solid preparation according to claim 3, wherein the filler is mannitol and microcrystalline cellulose.

14. The oral solid preparation according to claim 3, wherein the binder is hydroxypropyl cellulose.

15. The oral solid preparation according to claim 3, wherein the lubricant is magnesium stearate.

16. The oral solid preparation according to claim 1, wherein the L-HPC comprises 5.0% to 16.0% of a hydroxypropoxy group by dry weight.

17. The oral solid preparation according to claim 1, wherein the L-HPC is L-HPC LH-21.

18. An oral solid preparation comprising:
3% to 60% by weight of 4-hydroxy-6-{2-[4-(trifluoromethyl)phenyl]ethyl}pyridazine-3(2H)-one;
3% to 15% by weight of a L-HPC;
10% to 75% by weight of mannitol;
5% to 15% by weight of microcrystalline cellulose;
1% to 10% by weight of hydroxypropyl cellulose; and
0.2% to 3% by weight of magnesium stearate.

19. The oral solid preparation according to claim 1, further comprising a film coating.

20. The oral solid preparation according to claim 19, wherein the film coating comprises a coating agent and a coating additive.

21. The oral solid preparation according to claim 19, wherein the film coating comprises a coating agent and a light-shielding agent.

22. The oral solid preparation according to claim 19, wherein the film coating comprises a coating agent, a light-shielding agent, and a colorant.

23. The oral solid preparation according to claim 19, wherein the film coating comprises hydroxypropyl methylcellulose, titanium dioxide, and hydroxypropyl cellulose.

24. The oral solid preparation according to claim 1, wherein 70% or more of the DAAO inhibitor dissolves within 30 minutes when performing a first dissolution test using a first paddle method.

25. The oral solid preparation according to claim 24, wherein the first paddle method comprises paddling at 75 rpm using 900 mL of a 0.05 mol/L phosphate buffer solution (pH 6.8) comprising 0.05% of cetyltrimethylammonium bromide (CTAB).

26. The oral solid preparation according to claim 1, wherein 70% or more of the DAAO inhibitor dissolves within 30 minutes when performing a second dissolution test using a second paddle method, wherein the DAAO inhibitor was stored for two weeks at 40° C./90% relative humidity before performing the second dissolution test.

27. The oral solid preparation according to claim 26, wherein the second paddle method comprises paddling at 50 rpm using 900 mL of a 0.05 mol/L phosphate buffer solution (pH 6.8) containing 0.1% of sodium dodecyl sulfate (SDS).

28. A method for producing an oral solid preparation according to claim 1, comprising:
mixing a D-amino acid oxidase inhibitor and an additive to obtain a mixture;
granulating the mixture to obtain at least one granule;
mixing the at least one granule and a L-HPC to obtain at least one mixed granule; and
compressing the at least one mixed granule.

29. The method according to claim 28, wherein mixing the D-amino acid oxidase inhibitor and the additive further comprises mixing a L-HPC with the D-amino acid oxidase inhibitor and the additive.

30. A method for preventing or treating a disease preventable or treatable by a D-amino acid oxidase inhibitor, comprising administering an oral solid preparation according to claim 1 to a mammal in need thereof.

31. The method according to claim 30, wherein the disease preventable or treatable by a D-amino acid oxidase inhibitor is chosen from schizophrenia and other mental disorders, dementia and other cognitive disorders, anxiety disorder, mood disorders, sleep disorders, disorders generally first diagnosed in early childhood, childhood or adolescence, pain, neurodegenerative disorders, and ataxic disorders.
